(19) United States
(12) Patent Application Publication
(10) Pub. No.: US 2015/0167082 A1
(43) Pub. Date: Jun. 18, 2015

(54) GENOTYPING TEST FOR ASSESSING RISK OF AUTISM

(71) Applicant: Integragen, Evry (FR)

(72) Inventor: Jérôme Carayol, Montrouge (FR)

(21) Appl. No.: 14/384,058

(22) PCT Filed: Mar. 8, 2013
(86) PCT No.: PCT/EP2013/054757

§ 371 Date: Jan. 15, 2015

Related U.S. Application Data
(60) Provisional application No. 61/608,717, filed on Mar. 9, 2012.

(30) Foreign Application Priority Data
Mar. 9, 2012 (EP) 12305285.4

(51) Int. Cl. C12Q 1/68

(52) U.S. Cl. CPC C12Q 1/6883; C12Q 2600/156

(57) ABSTRACT

The invention relates to a method of determining a risk of, or of detecting the predisposition to or the presence of autism in a subject, the method comprising detecting the combined presence of risk-associated SNP alleles at multiple loci in a sample from said subject, which method comprises genotyping a single nucleotide polymorphism (SNP) in the gene loci of at least HTR5A, MACF1, RBFOX1, ABR, PTPRG, CACNA2D1, GFRA1, DSCAML1, CHRM3, LPPR4, DLG2, SLC9A9 and BASP1.

The present invention relates to a method of determining a risk of autism, or of detecting or the predisposition to or the presence of autism in a subject by detecting a combination of risk alleles in several genes simultaneously.

### BACKGROUND OF THE INVENTION

The Pervasive Developmental disorders (PDDs) referred here as “autism” are a heterogeneous group of disorders characterized by impairments in social interaction, deficits in verbal and nonverbal communication, restricted interests, and repetitive behaviors. The disorders included in the spectrum are Pervasive Developmental disorder, Not Otherwise Specified (PDD-NOS), Autistic disorder, Childhood Disintegrative disorder, Asperger syndrome, and Rett syndrome. Autism spectrum disorders (ASDs) represent three of the PDDs: Autistic disorder (AUT), Asperger syndrome (AS), and PDD-NOS.

The ASDs are currently diagnosed through clinical evaluation. Two standardized instruments are considered as “gold standards” in the diagnostic evaluation of autism: Autism Diagnostic Observation Schedule-Generic [ADOS-G] (Gotham et al. 2007) and the Autism Diagnostic Interview-Revised [ADI-R]) (Lord et al. 1994). The ADI-R is a semi-structured diagnostic interview conducted with parents that allows quantitative exploration of three domains altered in autism. It provides a diagnostic assessment from the age of 36 months. Only recently, a revised algorithm was published for young children aged 12-47 months (Kim and Lord 2012). The ADOS is a scale of observation of the child. It has been developed for children with language age equivalent of at least 36 months. A version for children aged less than 30 months with a mental age of at least 12 months has recently been developed: the ADOS-Toddler Module (Luyster et al. 2009). Those tools require training and are usually carried out by psychiatrists or psychologists.

Several screening-tools have been validated to date (Barton et al. 2011). Despite limited database regarding the psychometric properties of specific screeners, their value of screening is recognized. However, no specific screening tool for autism has been validated in children less than 12 months. For example, the M-CHAT (Robins et al., 2001), the most widely used, has been validated in children aged from 18 to 24 months.

The prevalence of ASDs has been recently estimated to 1 per 110 children in the US (Rice et al, 2009), making autism one of the most frequent childhood neuro developmental disorders, with males being more likely to have a diagnosis than females (male to female ratio of approximately 4:1). Autism has a strong genetic component, and siblings of autistic children have an increased risk of disease of approximately 19% (Ozonoff et al. 2011) compared to the prevalence. Monozygotic and dizygotic twin studies have shown that autism has a significant genetic component with monozygotic twin concordance rates estimated between 70-80% (Hallmayer et al, 2011). Autism does not follow a simple Mendelian inheritance pattern and this is thought to be due to the involvement of multiple genes (Veenstra-VanderWeele et al. 2004) with evidence for sex-specific risk alleles in autism (Stone et al. 2004).

Spontaneous mutations or rare inherited variants may help to explain etiology for a minority of cases, the inheritance pattern of common variants is likely central to disease risk in a majority of multiplex families.

There is no drug therapy available for ASDs, although some autistic individuals have been treated with anti-depressant drugs (e.g. fluoxetine) for secondary symptoms. The main treatments proposed are based on intensive educational programs. Applied early enough some studies show that as many as 50% of autistic children participating in those programs can be referred back to normal schooling and education. The age at which the therapy is proposed is of significant importance. Ideally the programs should start at 18 months age. However, if early symptoms and parental concerns at 12 and 18 months may be predictive of ASD diagnostic (Zwaigenbaum, 2010), the literature suggests that children do not receive a formal diagnosis of autism until the age of four (Shattuck et al., 2009, Chamak et al., 2011, CDC, 2012).

Several genes or SNPs associated with ASDs have been identified by academic groups and through in-house research efforts at IntegraGen SA (IntegraGen). For instance, Hussman et al, 2011 describes several hundreds of candidate genes for association to autism. Coutihno et al, 2007 analyzed the role in autism etiology of seven candidate genes in the serotonin metabolic and neurotransmission pathways and report a significant main effect of HTR5A in autism. Voineagu et al, 2011 and Martin et al, 2007 describe the neuronal specific splicing factor A2BP1 (also known as FOX1) as an autism susceptibility gene. Morrow et al, 2008 describes several known candidate genes associated to autism, as well as new candidate genes associated to autism, including PCDH10, DIA1 (c3orf58), NHE9 (SLC9A9), CNTN3, SCN7A and RNF8. Wang et al, 2009(a) describe 30 SNPs, located between genes CDH10 and CDH9 or in or bear other genes, as associated to autism. Weiss et al, 2009 describes several SNPs associated to autism, and involves gene SEMA5A as an autism susceptibility gene. Anney et al, 2010 discloses a SNP and 7 genes as associated to autism. WO2009/043178, WO2011/031786, and US2011/0207124 describe association to autism of various gene variants or SNPs. While these applications claim methods for diagnosing autism or risk of autism, no data demonstrating that a true diagnosis, with acceptable sensitivity, specificity, and positive and negative predictive values may be obtained by analyzing the disclosed gene variants or SNPs is presented. Only individual associations of gene variants or SNPs are described. Therefore, many genes or SNPs have been individually described as associated to autism or risk of autism. However, the contribution to disease risk of each individual gene identified is generally low, and the odds ratio per risk allele rarely is above 1.5. Thus, the predictive power for each gene individually is too small to be of clinical utility in complex diseases. In this respect, Abrahams et al, 2008, Voineagu et al, 2012, and Scherer et al, 2011 review the various methods for identifying genes or SNPs associated to autism and clearly highlight both the presence of genetic factors increasing the risk of autism and the high heterogeneity and complexity of these factors.

Even if the risk of autism associated to each SNP remains modest, the accumulation of multiple risk-associated alleles markedly increases the risk to develop autism (Carayol et al, 2010 and Carayol et al, 2011) supporting a polygenic component in autism. Such a polygenic model predicts that the more markers are used, the better autism could be predicted with the use of genetic scores that reflect the joint effect of multiple risk-associated SNPs.

A first multiple biomarker-based tool combining analysis of 4 distinct SNPs in 4 distinct genes (PITX1, ATP2B2, SLC25A12, and EN2) was developed and demonstrated to be able to estimate a predictive value of the risk to develop autism in siblings with unknown status of affected individuals (Carayol J et al, 2010 and US2011/0086777). A second multiple biomarker-based tool combining analysis of 8 distinct SNPs in 8 distinct genes, including 3 of genes of the previous test (PITX1, ATP2B2, EN2, JARID2MARK1, ITGB3, CNTNAP2, and HOXA1) was later developed and demonstrated to be able to estimate a predictive value of the risk to develop autism in siblings with unknown status of affected individuals (Carayol J et al, 2011 and WO2011/138372).

The ARISk® Familial Autism Panel, proposed by Transgenomic, Inc. in the USA, simultaneously tests eight SNPs in eight independent genes which have been shown to be associated with the development of autism.

However there is still a need for genetic tests with an improved predictive power that could be easily applied at any age and for pre-screening of individuals for eligibility for an ADI-R, thereby substantially shortening the time from diagnosis to treatment.

### SUMMARY OF THE INVENTION

The invention relates to a method of determining a risk of autism, or of detecting the predisposition to or the presence of autism in a subject, the method comprising detecting the combined presence of risk-associated single nucleotide polymorphism (SNP) alleles at multiple loci in a sample from said subject. The inventors have now identified a new set of genes, and more particularly a new set of SNPs, useful in a genetic test for determining whether an individual is at risk of autism.

The invention more particularly provides a method of determining a risk of autism, or of detecting predisposition to or the presence of autism in a subject, the method comprising genotyping a SNP in the gene loci of at least HTR5A, MACF1, RBFOX1, ABR, PTPRG, CACNA2D1, GFRA1, DSCAML1, CHRM3, LPPR4, DLG2, SLC9A9 and BASP1 in a sample from said subject.

In a particular embodiment, the method further comprises genotyping a SNP in the gene loci of any or all of KCNIP1, UGCG, NTRK3, PLCB1, NELL1, GPR98, MAGI2, PLAGL1, CNTN6, DLG4, ERC2, TRIM9, SYT14, JARID2, CDH13, SULF2, GRIN2A and NRG3, or combinations thereof.

In a another particular embodiment, the method further comprises genotyping a SNP in the gene loci of any or all of NRG1, TRIM2, EPHA5, PCDH10, HIP1, APBA1, PDE4D and EGLN3, or combinations thereof.

In a preferred embodiment, the method further comprises the additional genotyping of at least one SNP in the gene loci selected from the group consisting of ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, or combinations thereof.

In particular, the method may comprise genotyping of at least one SNP in all of the following the gene loci: ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG.

In the above methods, preferably, the SNP in HTR5A is rs893109 (position 27 of SEQ ID NO: 31), MACF1 is rs260969 (position 27 of SEQ ID NO: 15), RBFOX1 is rs12925135 (position 27 of SEQ ID NO: 39), ABR is rs2663327 (position 27 of SEQ ID NO: 40), PTPRG is rs636624 (position 27 of SEQ ID NO: 22), CACNA2D1 is rs2367910 (position 27 of SEQ ID NO: 41), GFRA1 is rs10787637 (position 27 of SEQ ID NO: 4), DSCAML1 is rs695083 (position 27 of SEQ ID NO: 24), CHRM3 is rs10802802 (position 27 of SEQ ID NO: 5), LPPR4 is rs712886 (position 27 of SEQ ID NO: 27), DLG2 is rs12275631 (position 27 of SEQ ID NO: 51), SLC9A9 is rs3928471 (position 27 of SEQ ID NO: 19), BASP1 is rs298542 (position 27 of SEQ ID NO: 52), KCNIP1 is rs12514116 (position 27 of SEQ ID NO: 38), UGCG is rs16916456 (position 27 of SEQ ID NO: 11), NTRK3 is rs7172184 (position 27 of SEQ ID NO: 28), PLCB1 is rs8123323 (position 27 of SEQ ID NO: 37), NELL1 is rs10766739 (position 27 of SEQ ID NO: 3), GPR98 is rs16868972 (position 27 of SEQ ID NO: 42), MAGI2 is rs12535987 (position 27 of SEQ ID NO: 43), PLAGL1 is rs2076683 (position 27 of SEQ ID NO: 12), CNTN6 is rs9837484 (position 27 of SEQ ID NO: 35), DLG4 is rs314253 (position 27 of SEQ ID NO: 17), ERC2 is rs1485677 (position 27 of SEQ ID NO: 8), TRIM9 is rs10150121 (position 27 of SEQ ID NO: 1), SYT14 is rs7534723 (position 27 of SEQ ID NO: 30), JARID2 is rs9370809 (position 27 of SEQ ID NO: 33), CDH13 is rs9940922 (position 27 of SEQ ID NO: 36), SULF2 is rs6063144 (position 27 of SEQ ID NO: 53), GRIN2A is rs4782109 (position 27 of SEQ ID NO: 21), NRG3 is rs2820100 (position 27 of SEQ ID NO: 54) or rs7075400 (position 27 of SEQ ID NO: 55), NRG1 rs723811 (position 27 of SEQ ID NO: 44), TRIM2 is rs11942354 (position 27 of SEQ ID NO: 45), EPHA5 is rs1597611 (position 27 of SEQ ID NO: 10), PCDH10 is rs4404561 (position 27 of SEQ ID NO: 20), HIP1 is rs6962352 (position 27 of SEQ ID NO: 25), APBA1 is rs11139294 (position 27 of SEQ ID NO: 6), PDE4D is rs35284 (position 27 of SEQ ID NO: 18), EGLN3 is rs946630 (position 27 of SEQ ID NO: 56), KCNH5 is rs1041644 (position 27 of SEQ ID NO: 2), MAP1S is rs12985015 (position 27 of SEQ ID NO: 7), GRM7 is rs1569284 (position 27 of SEQ ID NO: 9), PAX2 is rs2077642 (position 27 of SEQ ID NO: 13), PTPRD is rs2382104 (position 27 of SEQ ID NO: 14), PDE11A is rs2695112 (position 27 of SEQ ID NO: 16), RGS6 is rs6574041 (position 27 of SEQ ID NO: 23), ASTN2 is rs7021928 (position 27 of SEQ ID NO: 26), ACCN1 is rs7225320 (position 27 of SEQ ID NO: 29), DCLK2 is rs9307866 (position 27 of SEQ ID NO: 32), SLC24A2 is rs957910 (position 27 of SEQ ID NO: 34), AKAP7 is rs6923644 (position 27 of SEQ ID NO: 46), DCLK1 is rs1556060 (position 27 of SEQ ID NO: 47), MAP2K1 is rs1432443 (position 27 of SEQ ID NO: 48), CADM1 is rs220836 (position 27 of SEQ ID NO: 49), GRIN2B is rs7974275 (position 27 of SEQ ID NO: 50) and/or NAV2 is rs10500866 (position 27 of SEQ ID NO: 57). Most preferably, all SNPs genotyped are those mentioned in previous sentence.

The invention thus in particular provides a method of determining a risk of autism, or of detecting the predisposition to or presence of autism in a subject, the method comprising genotyping of SNPs in a sample from said subject, wherein said SNPs are rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456.

The method may also further comprise genotyping a SNP in the gene loci of any or all of PITX1, ATP2B2, EN2, JARID2, MARK1, ITGB3, CNTNAP2, and HOXA1, or combinations thereof, preferably the method further comprises genotyping any or all of the SNP selected from the group consisting rs6872664, rs2278556, rs1861972, rs7766973, rs12410279, rs5918, rs7794745, and rs10951154, or combinations thereof.

The invention further provides a method of determining a risk of autism, or of detecting the predisposition or presence of autism in a male subject, the method comprising genotyping a SNP in the gene loci of at least HTR5A, MACF1, RBFOX1, ABR, PTPRG, and CACNA2D1, in a sample from said subject. Preferably, the SNP in HTR5A is rs893109 (position 27 of SEQ ID NO: 31), in MACF1 is rs260969 (position 27 of SEQ ID NO: 15), in RBFOX1 is rs12925135 (position 27 of SEQ ID NO: 39), in ABR is rs2663327 (position 27 of SEQ ID NO: 40), in PTPRG is rs636624 (position 27 of SEQ ID NO: 22), and/or in CACNA2D1 is rs2367910 (position 27 of SEQ ID NO: 41). Most preferably, all SNPs genotyped are those mentioned in previous sentence. The invention thus in particular provides a method of determining a risk of autism, or of detecting the predisposition or presence of autism in a male subject, the method comprising genotyping of SNPs in a sample from said subject, wherein said SNPs are rs893109, rs260969, rs12925135, rs2663327, rs636624 and rs2367910.

Preferably, the method further comprises genotyping a SNP in the gene loci of any or all of KCNIP1, UGCG, NTRK3, PLCB1, NELL1, GPR98, MAGI2, and PLAGL1, or combinations thereof. In this case, advantageously, the SNP in KCNIP1 is rs12514116 (position 27 of SEQ ID NO: 38), in UGCG is rs16916456 (position 27 of SEQ ID NO: 11), in NTRK3 is rs7172184 (position 27 of SEQ ID NO: 28), in PLCB1 is rs8123323 (position 27 of SEQ ID NO: 37), in NELL1 is rs10766739 (position 27 of SEQ ID NO: 3), in GPR98 is rs16868972 (position 27 of SEQ ID NO: 42), in MAGI2 is rs12535987 (position 27 of SEQ ID NO: 43), and/or in PLAGL1 is rs2076683 (position 27 of SEQ ID NO: 12). Most preferably, all SNPs genotyped are those mentioned in previous sentence. Thus, preferably, the method further comprises genotyping any or all of the SNP selected from the group consisting rs12514116, rs16916456, rs7172184, rs8123323, rs10766739, rs16868972, rs12535987 and rs2076683, or combinations thereof.

More preferably, the method further comprises genotyping a SNP in the gene loci of any or all of NRG1, TRIM2, EPHA5, PCDH10, and HIP1, or combinations thereof. In this case, advantageously, the SNP in NRG1 is rs723811 (position 27 of SEQ ID NO: 44), in TRIM2 is rs11942354 (position 27 of SEQ ID NO: 45), in EPHA5 is rs1597611 (position 27 of SEQ ID NO: 10), in PCDH10 is rs4404561 (position 27 of SEQ ID NO: 20), and/or in HIP1 is rs6962352 (position 27 of SEQ ID NO: 25). Most preferably, all SNPs genotyped are those mentioned in previous sentence. Thus, the method preferably further comprises genotyping any or all of the SNP selected from the group consisting of rs723811, rs11942354, rs1597611, rs4404561 and rs6962352, or combinations thereof.

Even more preferably, the method further comprises genotyping a SNP in the gene loci of any or all of PDE11A, AKAP7, DCLK1, KCNH5, GRIN2A, ACCN1, DCLK2, ASTN2, GRM7, MAP2K1, CADM1, and GRIN2B, or combinations thereof. In this case, advantageously, the SNP in PDE11A is rs2695112 (position 27 of SEQ ID NO: 16), in AKAP7 is rs6923644 (position 27 of SEQ ID NO: 46), near 3′ of DCLK1 is rs1556060 (position 27 of SEQ ID NO: 47), in KCNH5 is rs1041644 (position 27 of SEQ ID NO: 2), in GRIN2A is rs4782109 (position 27 of SEQ ID NO: 21), in ACCN1 is rs7225320 (position 27 of SEQ ID NO: 29), in DCLK2 is rs9307866 (position 27 of SEQ ID NO: 32), in ASTN2 is rs7021928 (position 27 of SEQ ID NO: 26), in GRM7 is rs1569284 (position 27 of SEQ ID NO: 9), in MAP2K1 is rs1432443 (position 27 of SEQ ID NO: 48), in CADM1 is rs220836 (position 27 of SEQ ID NO: 49), and/or in GRIN2B is rs7974275 (position 27 of SEQ ID NO: 50). Most preferably, all SNPs genotyped are those mentioned in previous sentence. Thus, the method preferably further comprises genotyping any or all of the SNP selected from the group consisting of rs2695112, rs6923644, rs1556060, rs1041644, rs4782109, rs7225320, rs9307866, rs7021928, rs1569284, rs1432443, rs220836, and rs7974275, or combinations thereof.

In a preferred embodiment, the method further provides a method of determining a risk of autism, or of detecting the predisposition or presence of autism in a male subject, the method comprising genotyping any SNP or any combination of SPNs as identified in Table 1 or in Table 5.

The method may also further comprise genotyping a SNP in the gene loci of any or all of PITX1, ATP2B2, EN2, JARID2, CNTNAP2, and HOXA1, or combinations thereof, preferably the method further comprises genotyping any or all of the SNP selected from the group consisting rs6872664, rs2278556, rs1861972, rs7766973, rs7794745, and rs10951154, or combinations thereof.

The invention further provides a method of determining a risk of autism, or of detecting the predisposition or presence of autism in a female subject, the method comprising genotyping a SNP in the gene loci of at least CHRM3, DSCAML1, PTPRG, GFRA1, LPPR4, DLG2, SLC9A9 and BASP1, in a sample from said subject. Preferably, the SNP in CHRM3 is rs10802802 (position 27 of SEQ ID NO: 5), in DSCAML1 is rs695083 (position 27 of SEQ ID NO: 24), in PTPRG is rs636624 (position 27 of SEQ ID NO: 22), in LPPR4 is rs712886 (position 27 of SEQ ID NO: 27), in DLG2 is rs12275631 (position 27 of SEQ ID NO: 51), in SLC9A9 is rs3928471 (position 27 of SEQ ID NO: 19), in BASP1 is rs298542 (position 27 of SEQ ID NO: 52). Most preferably, all SNPs genotyped are those mentioned in previous sentence. The invention thus further provides a method of determining a risk of autism, or of detecting the predisposition to or presence of autism in a female subject, the method comprising genotyping of SNPs in a sample from said subject, wherein said SNPs are rs10802802, rs695083, rs636624, rs10787637, rs712886, rs12275631, rs3928471 and rs298542.

Preferably, the method further comprises genotyping a SNP in the gene loci of any or all of CNTN6, NTRK3, DLG4, ERC2, TRIM9, SYT14, JARID2, CDH13, SULF2, GRIN2A and NRG3, or combinations thereof. In this case, advantageously, the SNP in CNTN6 is rs9837484 (position 27 of SEQ ID NO: 35), in NTRK3 is rs7172184 (position 27 of SEQ ID NO: 28), in DLG4 is rs314253 (position 27 of SEQ ID NO: 17), in ERC2 is rs1485677 (position 27 of SEQ ID NO: 8), in TRIM9 is rs10150121 (position 27 of SEQ ID NO: 1), in SYT14 is rs7534723 (position 27 of SEQ ID NO: 30), in JARID2 is rs9370809 (position 27 of SEQ ID NO: 33), in CDH13 is rs9940922 (position 27 of SEQ ID NO: 36), in SULF2 is rs6063144 (position 27 of SEQ ID NO: 53), in GRIN2A is rs4782109 (position 27 of SEQ ID NO: 21), and/or in NRG3 is rs2820100 (position 27 of SEQ ID NO: 54) or rs7075400 (position 27 of SEQ ID NO: 55). Most preferably, all SNPs genotyped are those mentioned in previous sentence. Thus, preferably, the method further comprises genotyping any or all of the SNP selected from the group consisting of rs9837484, rs7172184, rs314253, rs1485677, rs10150121, rs7534723, rs9370809, rs9940922, rs6063144, rs4782109 and rs2820100, or combinations thereof.

More preferably, the method further comprises genotyping a SNP in the gene loci of any or all of APBA1, ABR, NRG3, PDE4D and EGLN3, or combinations thereof. In this case, advantageously, the SNP in APBA1 is rs11139294 (position 27 of SEQ ID NO: 6), in ABR is rs2663327 (position 27 of SEQ ID NO: 40), in NRG3 is rs7075400 (position 27 of SEQ ID NO: 55), in PDE4D is rs35284 (position 27 of SEQ ID NO: 18), and/or in EGLN3 is rs946630 (position 27 of SEQ ID NO: 56). Most preferably, all SNPs genotyped are those mentioned in previous sentence. Thus, the method preferably further comprises genotyping any or all of the SNP selected from the group consisting of rs11139294, rs2663327, rs7075400, rs35284 and rs946630, or combinations thereof.

Even more preferably, the method further comprises genotyping a SNP in the gene loci of any or all of RGS6, SLC24A2, PTPRD, NAV2, PCDH10, MAP1S, and PAX2, or combinations thereof. In this case, advantageously, the SNP in RGS6 is rs6574041 (position 27 of SEQ ID NO: 23), in SLC24A2 is rs957910 (position 27 of SEQ ID NO: 34), in PTPRD is rs2382104 (position 27 of SEQ ID NO: 14), in NAV2 is rs10500866 (position 27 of SEQ ID NO: 57), in PCDH10 is rs4404561 (position 27 of SEQ ID NO: 20), in MAP1S is rs12985015 (position 27 of SEQ ID NO: 7), and/or in PAX2 is rs2077642 (position 27 of SEQ ID NO: 13). Most preferably, all SNPs genotyped are those mentioned in previous sentence. Thus, the method preferably further comprises genotyping any or all of the SNP selected from the group consisting of rs6574041, rs957910, rs2382104, rs10500866, rs4404561, rs12985015, and rs2077642, or combinations thereof.

In a preferred embodiment, the method further provides a method of determining a risk of autism, or of detecting the predisposition or presence of autism in a female subject, the method comprising genotyping any SNP as identified in Table 1 or in Table 6.

The method may also further comprise genotyping a SNP in the gene loci of any or all of EN2, JARID2, MARK1, ITGB3, and CNTNAP2, or combinations thereof, preferably the method further comprises genotyping any or all of the SNP selected from the group consisting rs1861972, rs7766973, rs12410279, rs5918, and rs7794745, or combinations thereof.

In the methods of the invention, detecting the combined presence of risk-associated alleles, preferably as defined in Table 1, is indicative of a risk of autism, a predisposition to autism, or presence of autism in a subject. The level of risk or the likelihood of predisposition or presence of autism is determined depending on the number of risk-associated alleles that are detected, preferably by calculating a genetic score, as described in the Experimental section.

In one embodiment, the method of the invention comprises, or further comprises, genotyping any SNP in linkage disequilibrium with any of the SNP identified above, wherein said SNP in linkage disequilibrium is within the gene of said SNP identified above. In particular, the presence of SNPs in linkage disequilibrium (LD) with the above identified SNPs may be genotyped, in place of, or in addition to, said identified SNPs. In the context of the present invention, the SNPs in linkage disequilibrium with the above identified SNP are within the same gene of the above identified SNP.

The invention further provides a kit comprising primers pairs (forward and reverse primers) or triplets (two forward and one reverse primers) and/or probes for the specific detection of a SNP in the gene loci of at least HTR5A, MACF1, RBFOX1, ABR, PTPRG, CACNA2D1, GFRA1, DSCAML1, CHRM3, LPPR4, DLG2, SLC9A9 and BASP1, preferably the SNPs are rs893109 in HTR5A (position 27 of SEQ ID NO: 31), rs260969 in MACF1 (position 27 on SEQ ID NO: 15), rs12925135 in RBFOX1 (position 27 of SEQ ID NO: 39), rs2663327 in ABR (position 27 of SEQ ID NO: 40), rs636624 in PTPRG (position 27 of SEQ ID NO: 22), rs2367910 in CACNA2D1 (position 27 of SEQ ID NO: 41), rs10787637 in GFRA1 (position 27 of SEQ ID NO: 4), rs695083 in DSCAML1 (position 27 of SEQ ID NO: 24), rs10802802 in CHRM3 (position 27 of SEQ ID NO: 5), rs712886 in LPPR4 (position 27 of SEQ ID NO: 27), rs12275631 in DLG2 (position 27 of SEQ ID NO: 51), rs3928471 in SLC9A9 (position 27 of SEQ ID NO: 19), and rs298542 in BASP1 (position 27 of SEQ ID NO: 52).

The kit may further comprise primers pairs (forward and reverse primers) or triplets (two forward and one reverse primers) and/or probes for the specific detection of a SNP in the gene loci of any or all of KCNIP1, UGCG, NTRK3, PLCB1, NELL1, GPR98, MAGI2, PLAGL1, CNTN6, DLG4, ERC2, TRIM9, SYT14, JARID2, CDH13, SULF2, GRIN2A and NRG3, or combinations thereof, preferably the kit further comprises primers pairs (forward and reverse primers) or triplets (two forward and one reverse primers) and/or probes for the specific detection of any or all of rs12514116 in KCNIP1 (position 27 on SEQ ID NO: 38), rs16916456 in UGCG (position 27 of SEQ ID NO: 11), rs7172184 in NTRK3 (position 27 of SEQ ID NO: 28), rs8123323 in PLCB1 (position 27 of SEQ ID NO: 37), rs10766739 in NELL1 (position 27 of SEQ ID NO: 3), rs16868972 in GPR98 position 27 of SEQ ID NO: 42), rs12535987 in MAGI2 (position 27 of SEQ ID NO: 43), rs207668 in PLAGL1 (position 27 of SEQ ID NO: 12), rs9837484 in CNTN6 (position 27 of SEQ ID NO: 35), rs314253 in DLG4 (position 27 of SEQ ID NO: 17), rs1485677 in ERC2 (position 27 of SEQ ID NO: 8), rs10150121 in TRIM9 (position 27 of SEQ ID NO: 1), rs7534723 in SYT14 (position 27 of SEQ ID NO: 30), rs9370809 in JARID2 (position 27 of SEQ ID NO: 33), rs9940922 in CDH13 (position 27 of SEQ ID NO: 36), rs6063144 in SULF2 (position 27 of SEQ ID NO: 53), rs4782109 in GRIN2A (position 27 of SEQ ID NO: 21), and rs2820100 in NRG3 (position 27 of SEQ ID NO: 54), or combinations thereof.

Said kit may also or in addition further comprises primers pairs (forward and reverse primers) or triplets (two forward and one reverse primers) and/or probes for the specific detection of a SNP in the gene loci of any or all of NRG1, TRIM2, EPHA5, PCDH10, HIP1, APBA1, PDE4D and EGLN3, or combinations thereof, preferably the kit further comprises primers pairs (forward and reverse primers) or triplets (two forward and one reverse primers) and/or probes for the specific detection of any or all of rs723811 in NRG1 (position 27 on SEQ ID NO: 44), rs11139294 in APBA1 (position 27 of SEQ ID NO: 6), rs11942354 in TRIM2 (position 27 of SEQ ID NO: 45), rs1597611 in EPHA5 (position 27 of SEQ ID NO: 10), rs4404561 in PCDH10 (position 27 of SEQ ID NO: 20), rs6962352 in HIP1 (position 27 of SEQ ID NO: 25), rs7075400 in NRG3 (position 27 of SEQ ID NO: 55), rs35284 in PDE4D (position 27 of SEQ ID NO: 18) and rs946630 in EGLN3 (position 27 of SEQ ID NO: 56), or combinations thereof.

Said kit may also or in addition further comprises primers pairs (forward and reverse primers) or triplets (two forward and one reverse primers) and/or probes for the specific detection of at least one SNP in the gene loci selected from the group consisting of ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, or combinations thereof, preferably the kit further comprises primers pairs (forward and reverse primers) or triplets (two forward and one reverse primers) and/or probes for the specific detection of any or all of rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456, or combinations thereof.

In particular, the kit may comprise primers pairs (forward and reverse primers) or triplets (two forward and one reverse primers) and/or probes for the specific detection of at least one SNP in all of the following the gene loci: ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, preferably the kit comprises primers pairs (forward and reverse primers) or triplets (two forward and one reverse primers) and/or probes for the specific detection of all following SNPs: rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456.

The kit may also further comprise primers pairs (forward and reverse primers) or triplets (two forward and one reverse primers) and/or probes for the specific detection of a SNP in the gene loci of any or all of PITX1, ATP2B2, EN2, JARID2, MARK1, ITGB3, CNTNAP2, and HOXA1, or combinations thereof, preferably the kit further comprises primers pairs (forward and reverse primers) or triplets (two forward and one reverse primers) and/or probes for the specific detection of any or all of the SNP selected from the group consisting rs6872664, rs2278556, rs1861972, rs7766973, rs12410279, rs5918, rs7794745, and rs10951154, or combinations thereof.

Primer pairs (forward and reverse primers) or triplets (two forward and one reverse primers) may be used for specific amplification of part of a target gene comprising the SNP of interest. When only two primers are used, they are generally located each on one side of the target SNP of interest and are used in order to increase the amount of target sequence for further analysis. When three primers are used, the single reverse primer is preferably located on one side of the target SNP of interest, while the two corresponding forward primers are respectively specific of the protective or risk-associated allele of the SNP. The base differing between the two primers is preferably located in 3′ of the forward primers. Primers are polynucleotides of about 15 to about 25 nucleotides, preferably of about 18 to about 22 nucleotides.

A probe for the specific detection of a SNP in a gene locus may notably comprise or consist of a polynucleotide comprising at least 10 contiguous bases, preferably about 10 to about 60 bases, complementary to part of a target gene comprising the SNP of interest.

In particular, the invention provides a set of polynucleotides comprising at least 10 contiguous bases, preferably about 10 to about 60 bases, of (i) SEQ ID NO: 31, 15, 39, 40, 22, 41, 4, 24, 5, 27, 51, 19 and 52 respectively around position 27 of SEQ ID NO: 31, position 27 of SEQ ID NO:15, position 27 of SEQ ID NO: 39, position 27 of SEQ ID NO: 40, position 27 of SEQ ID NO: 22, position 27 of SEQ ID NO: 41, position 27 of SEQ ID NO: 4, position 27 of SEQ ID NO: 24, position 27 of SEQ ID NO:5, position 27 of SEQ ID NO: 27, position 27 of SEQ ID NO: 51 and position 27 of SEQ ID NO: 19, or (ii) of the complement of said sequences. Such a set of polynucleotides may further comprise polynucleotides comprising at least 10 contiguous bases, preferably about 10 to about 60 bases, of (i) SEQ ID NO:38, SEQ ID NO:11, SEQ ID NO:28, SEQ ID NO:37, SEQ ID NO:3, SEQ ID NO:42, SEQ ID NO:43, SEQ ID NO:12, SEQ ID NO:35, SEQ ID NO:17, SEQ ID NO:8, SEQ ID NO:1, SEQ ID NO:30, SEQ ID NO:33, SEQ ID NO:36, SEQ ID NO:53, SEQ ID NO:21, SEQ ID NO:54 and SEQ ID NO:55, respectively around positions of SEQ ID NO:38, SEQ ID NO:11, SEQ ID NO:28, SEQ ID NO:37, SEQ ID NO:3, SEQ ID NO:42, SEQ ID NO:43, SEQ ID NO:12, SEQ ID NO:35, SEQ ID NO:17, SEQ ID NO:8, SEQ ID NO:1, SEQ ID NO:30, SEQ ID NO:33, SEQ ID NO:36, SEQ ID NO:53, SEQ ID NO:21, SEQ ID NO:54 and SEQ ID NO:55 mentioned in Table 1, or (ii) of the complement of said sequences. Such a set of polynucleotides may further comprise polynucleotides comprising at least 10 contiguous bases, preferably about 10 to about 60 bases, of (i) SEQ ID NO:44, SEQ ID NO:45, SEQ ID NO:10, SEQ ID NO:20, SEQ ID NO:25, SEQ ID NO:6, SEQ ID NO:18 and SEQ ID NO:56, respectively around positions of SEQ ID NO:44, SEQ ID NO:45, SEQ ID NO:10, SEQ ID NO:20, SEQ ID NO:25, SEQ ID NO:6, SEQ ID NO:18 and SEQ ID NO:56 mentioned in Table 1, or (ii) of the complement of said sequences.

In a preferred embodiment, the invention provides a set of polynucleotides comprising at least 10 contiguous bases, preferably about 10 to about 60 bases, of (i) each of SEQ ID NO:1 to SEQ ID NO:57, respectively around positions of SEQ ID NO:1 to SEQ ID NO:57 mentioned in Table 1, or (ii) of the complement of said sequences.

The above sets of polynucleotides may further comprise at least 10 contiguous bases, preferably about 10 to about 60 bases, of (i) each of SEQ ID NO:58 to SEQ ID NO:65, respectively around positions of SEQ ID NO:58 to SEQ ID NO:65 mentioned in Table 2, or (ii) of the complement of said sequences.

A further subject of the invention is a microarray comprising a set of polynucleotides and optionally, a substrate on which the set of polynucleotides is immobilized, wherein the set of polynucleotides is as defined above.

The inventors showed that the predictive value that is obtained by detecting combinations of risk-associated alleles of polymorphisms in these genes is superior to the predictive value obtained when considering these risk-associated alleles individually, demonstrating its clinical validity. Genotyping these SNPs thus allows the estimation of a predictive value for the risk of developing ASDs, not only in yet non-diagnosed siblings of affected individuals, but more generally to any individual, in particular any child.

The clinical utility of this test resides in its ability to select at risk individuals for earlier down-stream diagnosis using psychological profiling tests (e.g. ADI-R or ADOS). The test may also be used in affected individuals to accompany these profiling tests to substantiate the diagnosis for ASDs and distinguish it from other psychiatric conditions.

The present invention further relates to methods for treating or preventing autism in a subject, the method comprising:
a) determining a risk of autism, or detecting predisposition to or the presence of autism in a subject by any method according to the invention described herein, and
b) if said subject is determined to be at risk of autism, as predisposed to autism or as suffering from autism, then submitting said subject to:
   i) a behavioral autism instrument, such as Autism Diagnostic Observation Schedule-Generic [ADOS-G],
   ii) an indirect, interview-based autism instrument with third parties, such as Autism Diagnostic Interview-Revised [ADI-R], and/or
   iii) Early Intensive Behavioural Intervention (EIBI).

Preferably, if the subject is determined to be at risk of autism, as predisposed to autism or as suffering from autism, then said subject is first rapidly submitted to a behavioral or an indirect, interview-based autism test, preferably the Autism Diagnostic Interview-Revised [ADI-R] test in order to confirm the diagnosis of autism. If autism diagnosis is confirmed, then the subject is rapidly submitted to Early Intensive Behavioural Intervention (EIBI), since early intervention has been found to improve outcome for autistic subjects.

### DETAILED DESCRIPTION OF THE INVENTION

Unless otherwise specified, the term “autism” refers to Autism Spectrum Disorders (ASDs) which is a heterogeneous group of disorders characterized by impairments in social interaction, deficits in verbal and nonverbal communication, and restricted repetitive and stereotyped patterns of behavior, interests and activities. Autism Spectrum Disorders (ASDs) are preferably targeted, they include the typical form of autism, Autistic disorder (AUT), and forms differing by the age of beginning, the number and the distribution of the autistic key symptoms, such as Asperger syndrome (AS), childhood disintegrative disorder and PDD-NOS. The methods of the invention are more preferably intended for Autistic disorder (AUT).

The invention provides diagnostic screening methods based on a monitoring of several genes in a subject. The subject may be at early, pre-symptomatic stage, or late stage. The subject may be any human male or female, preferably a child or a young adult. The subject can be asymptomatic. The subject can have a family history of autism or not. The method of the invention is useful when the subject is a sibling of an individual with an autism-spectrum disorder, i.e. an individual already diagnosed with an autism spectrum disorder. However it may also be useful when the subject to test is not related to anyone with an autism-spectrum disorder.

The method of the invention can be performed at any age after birth and used to pre-screen individuals requiring further assessment with the ADI-R, shortening the time from diagnosis to intervention.

The diagnosis methods can be performed in vitro, ex vivo or in vivo, preferably in vitro or ex vivo. They use a sample from the subject. The sample may be any biological sample derived from a subject, which contains nucleic acids. Examples of such samples include fluids, tissues, cell samples, organs, biopsies, etc. Most preferred samples are blood, plasma, saliva, jugal cells, urine, seminal fluid, etc. A particularly preferred sample is saliva. The sample may be collected according to conventional techniques and used directly for diagnosis or stored. The sample may be treated prior to performing the method, in order to render or improve availability of nucleic acids or polypeptides for testing. Treatments include, for instance, lysis (e.g., mechanical, physical, chemical, etc.), centrifugation, etc. Also, the nucleic acids may be pre-purified or enriched by conventional techniques, and/or reduced in complexity. Nucleic acids may also be treated with enzymes or other chemical or physical treatments to produce fragments thereof. Considering the high sensitivity of the claimed methods, very small amounts of sample are sufficient to perform the assay.

The finding of a specific allele in the sample is indicative of the presence of a gene locus variant in the subject, which can be correlated to the presence, predisposition or stage of progression of autism. For example, an individual having a germ line mutation has an increased risk of developing autism. The determination of the presence of an altered gene locus in a subject also allows the design of appropriate therapeutic intervention, which is more effective and customized. Also, this determination at the pre-symptomatic level allows a preventive regimen to be applied.

### Risk-Associated Genes and SNP

The invention relates to a method of determining a risk of autism, or of detecting the predisposition to or the presence of autism in a subject, the method comprising detecting the combined presence of risk-associated single nucleotide polymorphism (SNP) alleles at multiple loci in a sample from said subject. The inventors have now identified a new set of genes, and more particularly a new set of SNPs, useful in a genetic test for determining whether an individual is at risk of autism. More specifically, the inventors showed that specific combinations of risk-associated alleles of selected SNPs allowed to obtain a predictive power that is clinically very useful for determining a risk of autism.

The invention more particularly provides a method of determining a risk of autism, or of detecting predisposition to or the presence of autism in a subject, the method comprising genotyping a SNP in the gene loci of at least HTR5A, MACF1, RBFOX1, ABR, PTPRG, CACNA2D1, GFRA1, DSCAML1, CHRM3, LPPR4, DLG2, SLC9A9 and BASP1 in a sample from said subject. In an embodiment, the method comprises genotyping the single nucleotide polymorphism (SNP) rs893109 in HTR5A (position 27 of SEQ ID NO: 31), and/or genotyping the single nucleotide polymorphism (SNP) rs260969 in MACF1 (position 27 on SEQ ID NO: 15), and/or genotyping the single nucleotide polymorphism (SNP) rs12925135 in RBFOX1 (position 27 of SEQ ID NO: 39), and/or genotyping the single nucleotide polymorphism (SNP) rs2663327 in ABR (position 27 of SEQ ID NO: 40), and/or genotyping the single nucleotide polymorphism (SNP) rs636624 in PTPRG (position 27 of SEQ ID NO: 22), and/or genotyping the single nucleotide polymorphism (SNP) rs2367910 in CACNA2D1 (position 27 of SEQ ID NO: 41), and/or genotyping the single nucleotide polymorphism (SNP) rs10787637 in GFRA1 (position 27 of SEQ ID NO: 4), and/or genotyping the single nucleotide polymorphism (SNP) rs695083 in DSCAML1 (position 27 of SEQ ID NO: 24), and/or genotyping the single nucleotide polymorphism (SNP) rs10802802 in CHRM3 (position 27 of SEQ ID NO: 5), and/or genotyping the single nucleotide polymorphism (SNP) rs712886 in LPPR4 (position 27 of SEQ ID NO: 27), and/or genotyping the single nucleotide polymorphism (SNP) rs12275631 in DLG2 (position 27 of SEQ ID NO: 51), and/or genotyping the single nucleotide polymorphism (SNP) rs3928471 in SLC9A9 (position 27 of SEQ ID NO: 19), and/or genotyping the single nucleotide polymorphism (SNP) rs298542 in BASP1 (position 27 of SEQ ID NO: 52). Preferably, all genotyped SNPs are those mentioned in previous sentence.

In a particular embodiment, the method further comprises genotyping a SNP in the gene loci of any or all of KCNIP1, UGCG, NTRK3, PLCB1, NELL1, GPR98, MAGI2, PLAGL1, CNTN6, DLG4, ERC2, TRIM9, SYT14, JARID2, CDH13, SULF2, GRIN2A and NRG3, or combinations thereof. In a preferred embodiment, the method further comprises genotyping any or all of the SNP rs12514116 in KCNIP1 (position 27 on SEQ ID NO: 38), the SNP rs16916456 in UGCG (position 27 of SEQ ID NO: 11), the SNP rs7172184 in NTRK3 (position 27 of SEQ ID NO: 28), the SNP rs8123323 in PLCB1 (position 27 of SEQ ID NO: 37), the SNP rs10766739 in NELL1 (position 27 of SEQ ID NO: 3), the SNP rs16868972 in GPR98 position 27 of SEQ ID NO: 42), the SNP rs12535987 in MAGI2 (position 27 of SEQ ID NO: 43), the SNP rs207668 in PLAGL1 (position 27 of SEQ ID NO: 12), the SNP rs9837484 in CNTN6 (position 27 of SEQ ID NO: 35), the SNP rs314253 in DLG4 (position 27 of SEQ ID NO: 17), the SNP rs1485677 in ERC2 (position 27 of SEQ ID NO: 8), the SNP rs10150121 in TRIM9 (position 27 of SEQ ID NO: 1), the SNP rs7534723 in SYT14 (position 27 of SEQ ID NO: 30), the SNP rs9370809 in JARID2 (position 27 of SEQ ID NO: 33), the SNP rs9940922 in CDH13 (position 27 of SEQ ID NO: 36), the SNP rs6063144 in SULF2 (position 27 of SEQ ID NO: 53), the SNP rs4782109 in GRIN2A (position 27 of SEQ ID NO: 21), and the SNP rs2820100 in NRG3 (position 27 of SEQ ID NO: 54). Preferably, all genotyped SNPs are those mentioned in previous sentence.

In another particular embodiment, the method further comprises genotyping a SNP in the gene loci of any or all of NRG1, TRIM2, EPHA5, PCDH10, HIP1, APBA1, PDE4D and EGLN3, or combinations thereof. In a another preferred embodiment, the method further comprises genotyping any or all of the SNP rs723811 in NRG1 (position 27 on SEQ ID NO: 44), the SNP rs11139294 in APBA1 (position 27 of SEQ ID NO: 6), the SNP rs11942354 in TRIM2 (position 27 of SEQ ID NO: 45), the SNP rs1597611 in EPHA5 (position 27 of SEQ ID NO: 10), the SNP rs4404561 in PCDH10 (position 27 of SEQ ID NO: 20), the SNP rs6962352 in HIP1 (position 27 of SEQ ID NO: 25), the SNP rs7075400 in NRG3 (position 27 of SEQ ID NO: 55), the SNP rs35284 in PDE4D (position 27 of SEQ ID NO: 18) and the SNP rs946630 in EGLN3 (position 27 of SEQ ID NO: 56). Preferably, all genotyped SNPs are those mentioned in previous sentence.

In a preferred embodiment, the method further comprises the additional genotyping of at least one SNP in the gene loci selected from the group consisting of ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, or combinations thereof.

More preferably, the method further comprises genotyping a SNP in the gene loci of any or all of KCNH5, MAP1S, GRM7, PAX2, PTPRD, PDE11A, RGS6, ASTN2, ACCN1, DCLK2, SLC24A2, AKAP7, DCLK1, MAP2K1, CADM1, and NAV2. In a still preferred embodiment, the method further comprises the additional genotyping of at least one SNP selected from the group consisting of KCNH5 is rs1041644 (position 27 of SEQ ID NO: 2), MAP1S is rs12985015 (position 27 of SEQ ID NO: 7), GRM7 is rs1569284 (position 27 of SEQ ID NO: 9), PAX2 is rs2077642 (position 27 of SEQ ID NO: 13), PTPRD is rs2382104 (position 27 of SEQ ID NO: 14), PDE11A is rs2695112 (position 27 of SEQ ID NO: 16), RGS6 is rs6574041 (position 27 of SEQ ID NO: 23), ASTN2 is rs7021928 (position 27 of SEQ ID NO: 26), ACCN1 is rs7225320 (position 27 of SEQ ID NO: 29), DCLK2 is rs9307866 (position 27 of SEQ ID NO: 32), SLC24A2 is rs957910 (position 27 of SEQ ID NO: 34), AKAP7 is rs6923644 (position 27 of SEQ ID NO: 46), DCLK1 is rs1556060 (position 27 of SEQ ID NO: 47), MAP2K1 is rs1432443 (position 27 of SEQ ID NO: 48), CADM1 is rs220836 (position 27 of SEQ ID NO: 49), GRIN2B is rs7974275 (position 27 of SEQ ID NO: 50) and NAV2 is rs10500866 (position 27 of SEQ ID NO: 57). Preferably, all genotyped SNPs are those mentioned in previous sentence.

In particular, the method may comprise genotyping of at least one SNP in all of the following the gene loci: ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG.

Preferably, the SNP in HTR5A is rs893109 (position 27 of SEQ ID NO: 31), MACF1 is rs260969 (position 27 of SEQ ID NO: 15), RBFOX1 is rs12925135 (position 27 of SEQ ID NO: 39), ABR is rs2663327 (position 27 of SEQ ID NO: 40), PTPRG is rs636624 (position 27 of SEQ ID NO: 22), CACNA2D1 is rs2367910 (position 27 of SEQ ID NO: 41), GFRA1 is rs10787637 (position 27 of SEQ ID NO: 4), DSCAML1 is rs695083 (position 27 of SEQ ID NO: 24), CHRM3 is rs10802802 (position 27 of SEQ ID NO: 5), LPPR4 is rs712886 (position 27 of SEQ ID NO: 27), DLG2 is rs12275631 (position 27 of SEQ ID NO: 51), SLC9A9 is rs3928471 (position 27 of SEQ ID NO: 19), BASP1 is rs298542 (position 27 of SEQ ID NO: 52), KCNIP1 is rs12514116 (position 27 of SEQ ID NO: 38), UGCG is rs16916456 (position 27 of SEQ ID NO: 11), NTRK3 is rs7172184 (position 27 of SEQ ID NO: 28), PLCB1 is rs8123323 (position 27 of SEQ ID NO: 37), NELL1 is rs10766739 (position 27 of SEQ ID NO: 3), GPR98 is rs16868972 (position 27 of SEQ ID NO: 42), MAGI2 is rs12535987 (position 27 of SEQ ID NO: 43), PLAGL1 is rs2076683 (position 27 of SEQ ID NO: 12), CNTN6 is rs9837484 (position 27 of SEQ ID NO: 35), DLG4 is rs314253 (position 27 of SEQ ID NO: 17), ERC2 is rs1485677 (position 27 of SEQ ID NO: 8), TRIM9 is rs10150121 (position 27 of SEQ ID NO: 1), SYT14 is rs7534723 (position 27 of SEQ ID NO: 30), JARID2 is rs9370809 (position 27 of SEQ ID NO: 33), CDH13 is rs9940922 (position 27 of SEQ ID NO: 36), SULF2 is rs6063144 (position 27 of SEQ ID NO: 53), GRIN2A is rs4782109 (position 27 of SEQ ID NO: 21), NRG3 is rs2820100 (position 27 of SEQ ID NO: 54) or rs7075400 (position 27 of SEQ ID NO: 55), NRG1 rs723811 (position 27 of SEQ ID NO: 44), TRIM2 is rs11942354 (position 27 of SEQ ID NO: 45), EPHA5 is rs1597611 (position 27 of SEQ ID NO: 10), PCDH10 is rs4404561 (position 27 of SEQ ID NO: 20), HIP1 is rs6962352 (position 27 of SEQ ID NO: 25), APBA1 is rs11139294 (position 27 of SEQ ID NO: 6), PDE4D is rs35284 (position 27 of SEQ ID NO: 18), EGLN3 is rs946630 (position 27 of SEQ ID NO: 56), KCNH5 is rs1041644 (position 27 of SEQ ID NO: 2), MAP1S is rs12985015 (position 27 of SEQ ID NO: 7), GRM7 is rs1569284 (position 27 of SEQ ID NO: 9), PAX2 is rs2077642 (position 27 of SEQ ID NO: 13), PTPRD is rs2382104 (position 27 of SEQ ID NO: 14), PDE11A is rs2695112 (position 27 of SEQ ID NO: 16), RGS6 is rs6574041 (position 27 of SEQ ID NO: 23), ASTN2 is rs7021928 (position 27 of SEQ ID NO: 26), ACCN1 is rs7225320 (position 27 of SEQ ID NO: 29), DCLK2 is rs9307866 (position 27 of SEQ ID NO: 32), SLC24A2 is rs957910 (position 27 of SEQ ID NO: 34), AKAP7 is rs6923644 (position 27 of SEQ ID NO: 46), DCLK1 is rs1556060 (position 27 of SEQ ID NO: 47), MAP2K1 is rs1432443 (position 27 of SEQ ID NO: 48), CADM1 is rs220836 (position 27 of SEQ ID NO: 49), GRIN2B is rs7974275 (position 27 of SEQ ID NO: 50) and/or NAV2 is rs10500866 (position 27 of SEQ ID NO: 57). Most preferably, all SNPs genotyped are those mentioned in previous sentence.

The invention thus in particular provides a method of determining a risk of autism, or of detecting the predisposition to or presence of autism in a subject, the method comprising genotyping of SNPs in a sample from said subject, wherein said SNPs are rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456.

Alternatively, the method may comprise genotyping at least one SNP as set forth in any of SEQ ID NO:1 to SEQ ID NO:57.

The method may also further comprise genotyping a SNP in the gene loci of any or all of PITX1, ATP2B2, EN2, JARID2, MARK1, ITGB3, CNTNAP2, and HOXA1, or combinations thereof, preferably the method further comprises genotyping any or all of the SNP selected from the group consisting rs6872664, rs2278556, rs1861972, rs7766973, rs12410279, rs5918, rs7794745, and rs10951154, or combinations thereof. These genes and SNPs correspond to those disclosed in Carayol et al, 2011 and in WO2011/138372. Indeed, the addition of these genes/SNPs to the genotyping further slightly improves the reliability of the test, as shown in the Examples.

In another embodiment, the presence of SNPs in linkage disequilibrium (LD) with the above identified SNPs may be genotyped, in place of, or in addition to, said identified SNPs. In the context of the present invention, the SNPs in linkage disequilibrium with the above identified SNP are within the same gene of the above identified SNP.

The method of the invention, also referred to as “the test” thus preferably includes genotyping all identified SNPs, or subcombinations thereof. The test can be used to strengthen the diagnosis by confirming a known risk profile. In such case a negative test result does not invalidate the diagnosis for autism.

Alternatively the test can be used to establish a detailed risk profile for a non-diagnosed patient, who may be a sibling of an individual diagnosed with autism, or not. A possible outcome is defined as the presence of a risk allele in one or more SNPs, in a heterozygous or homozygous status, implicating increased risk.

Following Table 1 describes the SNPs and their risk-/protective-associated alleles identified as being useful in the present invention, in combination or in subcombinations.

Following Table 2 describes the SNPs and their risk-/protective-associated alleles identified as being useful in Carayol et al, 2011 and in WO2011/138372.

TABLE 1
Autism-associated SNPs in combination

| | | Relative | | Pro- | | | | Position | |
|---|---|---|---|---|---|---|---|---|---|
| | | position | | tec- | | | | of SNP | Gender |
| | | to the | Risk | tive | | | SEQ | in SEQ | speci- |
| SNP | Gene | gene | Allele | Allele | dbSNP context sequence | Strand | ID NO: | ID NO: | ficity |
| rs10150121 | TRIM9 | intron | C | T | CTGTGCTACTTAATGTAGACCAC | + | 1 | 27 | All |
| | | | | | CTG[C/T]TTGATTTCCTGAATGTG | | | | |
| | | | | | GTCTATGT | | | | |
| rs1041644 | KCNH5 | intron | C | A | CACCATTTTAAAGAGTTTAACTA | − | 2 | 27 | All |
| | | | | | AAT[A/C]AAATTCATCAATGTTTC | | | | |
| | | | | | CACTATGT | | | | |
| rs10766739 | NELL1 | intron | A | G | ACACAATTGGCAAAACCCCCTGT | + | 3 | 27 | All |
| | | | | | CAC[A/G]GTCAGTAAACTTTGAG | | | | |
| | | | | | GACCTGCTC | | | | |
| rs10787637 | GFRA1 | intron | G | A | TGGATAGTTGGACTCTGCAACCT | + | 4 | 27 | All |
| | | | | | ACT[A/G]AAACAAACATGTTAAAA | | | | |
| | | | | | ATTAAACA | | | | |
| rs10802802 | CHRM3 | intron | G | A | TCTGTCTCATCCCTGGTCAGGAT | + | 5 | 27 | Female |
| | | | | | GTA[A/G]GTATAAGTTTGAAGGAT | | | | |
| | | | | | CAAAAAAT | | | | |
| rs11139294 | APBA1 | intron | G | A | TGCCCTCATGGAACTTACCCTGA | + | 6 | 27 | All |
| | | | | | GGA[A/G]TTTACAATAGAAATAAT | | | | |
| | | | | | TAAACATA | | | | |
| rs12985015 | MAP1S | 5′ | G | A | CAGCAGTCCTGAGGGCTCAGGG | + | 7 | 27 | All |
| | | | | | TTCC[A/G]TTTTCCCCACAAA | | | | |
| | | | | | TGCCATCATCTG | | | | |
| rs1485677 | ERC2 | intron | G | A | TTTTAATTAATCCTCTGCAAGGA | + | 8 | 27 | All |
| | | | | | ACC[A/G]AGTTTTGTTTGCCAT | | | | |
| | | | | | CATCTCCCCA | | | | |
| rs1569284 | GRM7 | intron | G | A | AAAGTCTATTTATTTTCCCAACTA | − | 9 | 27 | All |
| | | | | | AT[A/G]TGTGTATGCTTCATGAG | | | | |
| | | | | | AGCACAGC | | | | |
| rs1597611 | EPHA5 | intron | G | A | AGTGAGAAGTTATGGTGCTTTCT | + | 10 | 27 | All |
| | | | | | CTC[A/G]CCTGCCTATGGCTGCC | | | | |
| | | | | | CACAGTCCA | | | | |
| rs16916456 | UGCG | intron | C | T | TGATGTCAGTGATTGTAACTGTC | + | 11 | 27 | Male |
| | | | | | ATT[C/T]CTAGAACTTGTGTGGTT | | | | |
| | | | | | CTTTTCAT | | | | |
| rs2076683 | PLAGL1 | intron | T | G | AATGAACTGACACTGCACAACAA | + | 12 | 27 | All |
| | | | | | GAC[G/T]GTCACATAAAACCACA | | | | |
| | | | | | GGAATCACT | | | | |
| rs2077642 | PAX2 | intron | T | C | CTCCTGTAGGAGGCTTGAGCCT | + | 13 | 27 | Female |
| | | | | | GGGT[C/T]TAGGTTGGAGACAGA | | | | |
| | | | | | GGCCGAGAAG | | | | |
| rs2382104 | PTPRD | intron | G | T | ATGGACAGCCTATAGGCTGTAA | + | 14 | 27 | All |
| | | | | | CCTG[G/T]TATTAGGAATAAAGCT | | | | |
| | | | | | TTCCCTATA | | | | |
| rs260969 | MACF1 | intron | T | C | GTAAACCCTCCTCCTCTTTTAAG | + | 15 | 27 | All |
| | | | | | TGC[C/T]GCCTCCTCTGTCCTTAA | | | | |
| | | | | | TGCCCCCA | | | | |
| rs2695112 | PDE11A | intron | A | G | TCATCCCCCCATGTCGACCTAAA | − | 16 | 27 | Female |
| | | | | | AGA[A/G]CACAGTTTACTTTTTCA | | | | |
| | | | | | AGGTTCTC | | | | |
| rs314253 | DLG4 | 3′ | G | A | AAGTTGAGAGTTTCATGCAAAAG | − | 17 | 27 | All |
| | | | | | ACC[A/G]ACCCAGGGGTAGTGAT | | | | |
| | | | | | TCTGTGGAT | | | | |
| rs35284 | PDE4D | intron | A | G | CGTTGTAATTCTATCTTCAGAAT | + | 18 | 27 | All |
| | | | | | GAT[A/G]CATTGCAAAAGAGTGT | | | | |
| | | | | | GACAAAAGG | | | | |
| rs3928471 | SLC9A9 | intron | T | C | GCTTCATGTTTATGTTCTATGTT | + | 19 | 27 | All |
| | | | | | CAG[C/T]TTTTGGTCTGTT | | | | |
| rs4404561 | PCDH10 | 5′ | C | A | TACCAGGGCCTGCTCAGCAACC | + | 20 | 27 | All |
| | | | | | AGAG[A/C]AGCAGAATGGGGGG | | | | |
| | | | | | CCAGATGCAAG | | | | |
| rs4782109 | GRIN2A | intron | T | C | ATAGCAATGATGGACAAACTCCC | + | 21 | 27 | All |
| | | | | | TGC[C/T]TCATGGGGCTTGCATT | | | | |
| | | | | | TTAGAGCTA | | | | |
| rs636624 | PTPRG | intron | A | G | TTGGTCACTGCCTATTTCAATTC | − | 22 | 27 | All |
| | | | | | TGG[A/G]TTTCTCTAACAATGAGA | | | | |
| | | | | | AATGGTCT | | | | |
| rs6574041 | RGS6 | intron | A | G | GTAGTAGGATGTTTGAAGAACA | + | 23 | 27 | All |
| | | | | | GCAA[A/G]GAGACCAGTGTGGCT | | | | |
| | | | | | GGAAGAAGGG | | | | |
| rs695083 | DSCAML1 | intron | T | C | ACTGGTTTGAGGTCTCCCCCTG | − | 24 | 27 | Female |
| | | | | | GAGC[C/T]ACCCAGAACACATCC | | | | |
| | | | | | AGGCCCTACC | | | | |
| rs6962352 | HIP1 | intron | G | A | GGGTAGCAGTGGCTGTCCCTGT | + | 25 | 27 | All |
| | | | | | GGGT[A/G]AAGTCACCTGACCAG | | | | |
| | | | | | CCACTGTGAG | | | | |
| rs7021928 | ASTN2 | intron | A | G | GAACTGTAGCAGGTTCTTTGACA | + | 26 | 27 | All |
| | | | | | TGT[A/G]TTATTTTATATCACAAC | | | | |
| | | | | | AAGCAGAG | | | | |
| rs712886 | LPPR4 | intron | C | T | TAGTGAATAAGAGATAGACTTGG | − | 27 | 27 | Female |
| | | | | | TCA[C/T]CAAGGAGCAATAGCTT | | | | |
| | | | | | AGTGAGAGA | | | | |
| rs7172184 | NTRK3 | intron | C | T | AGATCTAGCTCTCTCAGGCACAA | + | 28 | 27 | All |
| | | | | | ACA[C/T]CCAGATATTTTGTGATA | | | | |
| | | | | | GAAGGAAA | | | | |
| rs7225320 | ACCN1 | intron | T | C | TCCCCTTTCTTAAGGATACAGAC | + | 29 | 27 | All |
| | | | | | TTT[C/T]ATCAGCAGTGATCACTC | | | | |
| | | | | | ATTCACCA | | | | |
| rs7534723 | SYT14 | intron | A | G | TGTACTCTTGCATGAAACCAGGA | + | 30 | 27 | Female |
| | | | | | GAA[A/G]GTTTTACTTGGTTTGCT | | | | |
| | | | | | AAACTTTG | | | | |
| rs893109 | HTR5A | 3′ | G | A | AATATGCGAACTTTCACTTAAAA | + | 31 | 27 | Male |
| | | | | | AGT[A/G]GGGAAAATATAGGATC | | | | |
| | | | | | TCTGAATGC | | | | |
| rs9307866 | DCLK2 | intron | T | C | TAGTGCAAGAGGGGGATGTTTG | + | 32 | 27 | All |
| | | | | | GTAT[C/T]GTGGATTGCACAGTG | | | | |
| | | | | | ACCTTGTTTT | | | | |
| rs9370809 | JARID2 | intron | T | C | TTGGAGGGCATGCTGGTTGCAA | + | 33 | 27 | All |
| | | | | | CCCT[C/T]TTATTCTAATAAGGAA | | | | |
| | | | | | CTGGTTTGG | | | | |
| rs957910 | SLC24A2 | intron | T | C | CCTCCTGACAGTGTTGTGTCCT | − | 34 | 27 | All |
| | | | | | GTAA[C/T]TGAAAGAGGATTGCA | | | | |
| | | | | | TCTGCACTCA | | | | |
| rs9837484 | CNTN6 | intron | G | A | ACCGACATCAGTGGTCCATTCA | + | 35 | 27 | Female |
| | | | | | GTGG[A/G]CCATTAATGTTGCCT | | | | |
| | | | | | GACATTAAGT | | | | |
| rs9940922 | CDH13 | intron | G | A | TGTAGCCTCCAGGGTTGCTGTG | + | 36 | 27 | Female |
| | | | | | GAAG[A/G]GAAAGAGAGAGCAAG | | | | |
| | | | | | GAGGGTCTTG | | | | |
| rs8123323 | PLCB1 | intron | T | C | TGATTTGAACCGTCAATACCAAC | + | 37 | 27 | All |
| | | | | | CCC[C/T]TAACCCCAGTAAAAAAA | | | | |
| | | | | | AAAACAGC | | | | |
| rs12514116 | KCNIP1 | intron | C | T | TGGGGCCTCCTGGTGCTCCTCA | + | 38 | 27 | Male |
| | | | | | GAAT[C/T]ACGTGCTCTGGGCAG | | | | |
| | | | | | GAAAAAGGTG | | | | |
| rs12925135 | RBFOX1 | intron | C | T | TTTCTAATCCTTACCTCTCAGAG | + | 39 | 27 | Male |
| | | | | | GGA[C/T]GATTATGAGAAGGAAA | | | | |
| | | | | | TGAACTATC | | | | |
| rs2663327 | ABR | intron | C | T | CTTGTCTCTGCCCTGAAGCACA | − | 40 | 27 | All |
| | | | | | GCCA[C/T]GTGGGTCTGAGAATC | | | | |
| | | | | | CCCTACTTCC | | | | |
| rs2367910 | CACNA2D1 | intron | C | A | TTTTTAAATCTTTTTCTTTGTGAA | + | 41 | 27 | Male |
| | | | | | CA[A/C]GTAAATTGAAATGAAAAG | | | | |
| | | | | | CTGAGTA | | | | |
| rs16868972 | GPR98 | missense | T | G | AACATCACACTATCAATAATAAG | + | 42 | 27 | Male |
| | | | | | GTT[G/T]AAAGGCCTCATGGGAA | | | | |
| | | | | | AAGTCCTTG | | | | |
| rs12535987 | MAGI2 | intron | C | T | TAAGTTTTGTGGGCACTATTGAT | + | 43 | 27 | Male |
| | | | | | AAA[C/T]AAAATGAAAGATAAAGA | | | | |
| | | | | | CAAATGAA | | | | |
| rs723811 | NRG1 | intron | T | C | CTCACAATTCAATGTTTTAGCAT | − | 44 | 27 | Male |
| | | | | | ATA[C/T]CATCAGGCAAAACTATC | | | | |
| | | | | | AATTTTGA | | | | |
| rs11942354 | TRIM2 | near-gene-5 | A | G | GTGGCGGTGATTCCCAGGTCTG | + | 45 | 27 | Male |
| | | | | | GTTG[A/G]TCAAGACTGCAATGC | | | | |
| | | | | | ACAACAGGAA | | | | |
| rs6923644 | AKAP7 | intron | G | T | GGAAGTAATTGTATTGCATTAAT | + | 46 | 27 | Male |
| | | | | | CAG[G/T]ACCATTATTTAGTATTG | | | | |
| | | | | | GACATTTC | | | | |
| rs1556060 | DCLK1 | near-gene-3 | G | A | CTCACTCAACAGGTTTCAATGGG | − | 47 | 27 | Male |
| | | | | | GGA[A/G]CAAATAACAATACGCA | | | | |
| | | | | | AGGTTAATA | | | | |
| rs1432443 | MAP2K1 | intron | T | G | CCTTGTAACACTACAGAAGGATA | − | 48 | 27 | Male |
| | | | | | TGT[G/T]AGGATTAGAGAATTTTA | | | | |
| | | | | | GCACTGGA | | | | |
| rs220836 | CADM1 | intron | G | A | ATATATTTTACAGTAGTTGTCAAT | − | 49 | 27 | Male |
| | | | | | CT[A/G]TTTTCCAGTTTTTCTGGT | | | | |
| | | | | | ACTTTTT | | | | |
| rs7974275 | GRIN2B | intron | G | T | TAATGTAACAACCAACTGGTCTC | + | 50 | 27 | Male |
| | | | | | CAT[G/T]TCCTTATAGGATTAAAA | | | | |
| | | | | | GCTATTAA | | | | |
| rs12275631 | DLG2 | intron | T | C | ATATAGTGAGAAATATTACTGAT | + | 51 | 27 | Female |
| | | | | | GAG[C/T]GGACTGAAACTGTTCA | | | | |
| | | | | | TTGCATATT | | | | |
| rs298542 | BASP1 | intron | C | T | CAACGGGTAGGAAAGGACAGTT | + | 52 | 27 | Female |
| | | | | | GGTT[C/T]AGTGCTTGCTCATGTT | | | | |
| | | | | | AGCCCTGTA | | | | |
| rs6063144 | SULF2 | intron | C | T | ACTGCCGTAAATCACTGACTTTG | + | 53 | 27 | Female |
| | | | | | AGC[C/T]TCAGCTTCCCTGTCTG | | | | |
| | | | | | TAAAAACAC | | | | |
| rs2820100 | NRG3 | intron | C | A | ATGTCATTAGTCTTTGACCAATA | + | 54 | 27 | Female |
| | | | | | TTT[A/C]TCCAGTCCTTATCCAGC | | | | |
| | | | | | CCCAGTTC | | | | |
| rs7075400 | NRG3 | intron | T | C | GTTTGGGGAATATGTTTTTAGAA | + | 55 | 27 | Female |
| | | | | | ATA[C/T]ACATGCCATATGTGAG | | | | |
| | | | | | GCTATAGAA | | | | |
| rs946630 | EGLN3 | unknown | G | A | TCTTTGGGGAACTGAAAGAAGC | + | 56 | 27 | Female |
| | | | | | CCTG[A/G]AGAACACAATATACA | | | | |
| | | | | | ATGGCACACC | | | | |
| rs10500866 | NAV2 | intron | T | C | CAGAGCTGGGCATATACAGTAG | + | 57 | 27 | Female |
| | | | | | GAGA[C/T]GTTTGCTATATTTTAG | | | | |
| | | | | | GTAATTAAT | | | | |

<< All >> means a SNP associated to autism in males and females;
<< Males >> and << Females >> mean a SNP associated to autism in males or in females respectively.

TABLE 2
Autism-associated SNPs disclosed in Carayol et al, 2011 and in WO2011/138372

| | | | | Pro- | | | | Position | |
|---|---|---|---|---|---|---|---|---|---|
| | | Relative | | tec- | | | | of SNP | Gender |
| | | position | Risk | tive | | | SEQ | in SEQ | speci- |
| SNP | Gene | to the gene | Allele | Allele | dbSNP context sequence | Strand | ID NO: | ID NO: | ficity |
| rs6872664 | PITX1 | intron | C | T | TGCTTTTCTGAACTAGGATCA | + | 58 | 27 | Male |
| | | | | | GATCT[C/T]TCCAGCCTAAAG | | | | |
| | | | | | TCCCTCCACTTTC | | | | |
| rs2278556 | ATP2B2 | intron | A | G | TTACGTGCCTATCATCCAGCT | + | 59 | 27 | Male |
| | | | | | TTGTA[A/G]CATCTTAACATTA | | | | |
| | | | | | TGCCGTACTTGC | | | | |
| rs1861972 | EN2 | intron | A | G | AGAGGCGAGGTCACCACTCC | + | 60 | 27 | All |
| | | | | | CTGCCA[A/G]TGGCCTTGCCC | | | | |
| | | | | | CCTTCTTCCCCCAC | | | | |
| rs7766973 | JARID2 | intron | C | T | CCCAGAGGGTTTATATTTTAC | + | 61 | 27 | All |
| | | | | | CTGCA[C/T]TCCTGAGGATGT | | | | |
| | | | | | GTTTGTGTTGCTT | | | | |
| rs12410279 | MARK1 | 3′ | A | G | AGTACTGCAAAACAGGACAG | + | 62 | 27 | Female |
| | | | | | CCATCA[A/G]AGATTCTTCCCT | | | | |
| | | | | | GATGACATCTCAG | | | | |
| rs5918 | ITGB3 | intron | T | C | GGCTCCTGTCTTACAGGCCC | + | 63 | 27 | Female |
| | | | | | TGCCTC[C/T]GGGCTCACCTC | | | | |
| | | | | | GCTGTGACCTGAAG | | | | |
| rs7794745 | CNTNAP2 | intron | T | A | ACAGGTCAGGACCTGGAAAG | + | 64 | 27 | All |
| | | | | | GCCTAA[A/T]TGATAAGACTAA | | | | |
| | | | | | GTGTCAAAATCAG | | | | |
| rs10951154 | HOXA1 | exon | T | C | TCTGGTAGGTAGCCGGCTGG | + | 65 | 27 | Male |
| | | | | | GGGTGG[C/T]GATGGTGGTG | | | | |
| | | | | | GTGGTGGTGGTGGTG | | | | |

<< All >> means a SNP associated to autism in males and females;
<< Males >> and << Females >> mean a SNP associated to autism in males or in females respectively.

### Risk Determination

Once SNPs of interest have been genotyped, a risk of autism, a predisposition to or the presence of autism in the tested subject is determined.

In the methods of the invention, detecting the combined presence of risk-associated alleles, preferably as defined in Table 1, is indicative of a risk of autism, a predisposition to autism, or presence of autism in a subject. The risk level or the likelihood of predisposition or presence of autism is determined depending on the number of risk-associated alleles that are detected, preferably by calculating a genetic score. The genetic score (GS) is then compared to one or more threshold value(s).

A genetic score is first calculated based on the risk or protective nature of each genotyped SNP. Table 1 defines the risk and protective alleles of each of the specific 57 SNPs associated to autisms in the present invention.

A genetic score is calculated by making an optionally weighted sum of the risk-associated genotyped SNPs.

More precisely, when n distinct SNPs are genotyped, a genetic score may be calculated using the following formula:

$$GS = {\sum }_{i = 1}^{n} {x}_{i}$$

wherein each xᵢ, 1≦i≦n, is the weight of each genotyped SNPi.

Since any SNP will be genotyped for both alleles of the subject, the participation of each SNP to the genetic score may be weighted depending on the underlying genetic model of association of the SNP to autism.

Three genetic models are possible: an additive model, a recessive model and a dominant model. In a recessive model, only the presence of two risk alleles will impact the autism risk. In a dominant model, the presence of one or two risk alleles will similarly impact the autism risk. Finally, in an additive model, the presence of one risk allele will impact the autism risk, while the presence of a further second risk allele will further impact the autism risk.

Generally, an additive model is assumed as default model to modelize the genotype of individuals for each SNPs analyzed in an association study for statistical purpose (Pereira, Patsopoulos et al. 2009). Under this model, each tested SNPi participates to the genetic score as follows: xᵢ=0 for “no risk allele”, 1 xᵢ=1 for “one risk allele” and xᵢ=2 for “two risk alleles”. This case corresponds to the simpler genetic score, which then corresponds to the sum of risk alleles genotyped in the sample.

Such an additive default model may be used in the context of the present invention, and still permits reasonable reliability of the risk determination (see Examples). However, one way to weight the SNPs in the genetic score and to improve the reliability of the test consists in using their true underlying genetic model. When SNPi is recessive, it adds 0 point to the genetic score (xᵢ=0) if the individual is homozygous non carrier of the risk allele and heterozygous, and 2 points (xᵢ=2) if he is homozygous carrier of the risk allele. Similarly, when the SNP is dominant, it adds 0 point to the genetic score (xᵢ=0) if the individual is homozygous non carrier of the risk allele, and 2 points (xᵢ=2) otherwise.

The choice of the best genetic model for a given SNPi may be made based on analysis of a reference (training) population of samples, as described in the examples. For a proportion of SNPs, all three genetic models or two alternative genetic models may be used without significant impact on the reliability of the test.

The values of xᵢ depending on the selected genetic model and the number (0, 1 or 2) of risk-associated alleles genotyped are summarized in following Table 3.

TABLE 3
SNPi weight (xi) as function of the genetic model and number
(0, 1 or 2) of risk-associated alleles genotyped in the subject
sample. We assume that allele 2 is the risk allele for SNPi

| | Genotype “1 1” | Genotype “1 2” | Genotype “2 2” |
|---|---|---|---|
| | (homozygous | or “2 1” | (homozygous |
| Genetic model | protective allele) | (heterozygous) | risk allele) |
| Additive | 0 | 1 | 2 |
| Recessive | 0 | 0 | 2 |
| Dominant | 0 | 2 | 2 |

Another weighting consists in using odds ratios estimated for each genotype using the homozygous non carrier as reference: as described in Table 4 below where OR₁₁ equal 1 as the reference genotype, ORₕₑₜ is the odds ratio associated to the heterozygous genotype and ORₕₒₘ is the odds ratio associated to the homozygous carrier genotype. Odds ratio may be estimated using classical logistic regression in the discovery (training) population.

TABLE 4
SNPi weight (xᵢ) as function of the odds ratio.
We assume that allele 2 is the risk allele for SNPi
and genotype “1 1” is the reference genotype

| | | Genotype “1 2” | |
|---|---|---|---|
| Genetic model | Genotype “1 1” | or “2 1” | Genotype “2 2” |
| Odds ratio | 1 | ORₕₑₜ | ORₕₒₘ |

While this weighting may, contrary to the mere selection of an appropriate genetic model, take into account the fact that some SNPs may impact the autism risk more than others, as explained in the introduction, the contribution to disease risk of each individual SNP is generally low, and the use of weights based on odds ratio does not significantly improve the reliability of the test.

Therefore, advantageously, when n distinct SNPs are genotyped, a genetic score may be calculated using the following formula:

$$GS = {\sum }_{i = 1}^{n} {x}_{i}$$

wherein each xᵢ, 1≦i≦n, is the weight of each genotyped SNPi defined based on an additive, a recessive or a dominant genetic model (see Table 3). In an embodiment, each xᵢ, 1≦i≦n, is the weight of each genotyped SNPi defined based on an additive genetic model (see Table 3). In a preferred embodiment, each xᵢ, 1≦u≦n, is the weight of each genotyped SNPi defined based on an additive, a recessive or a dominant genetic model (see Table 3), wherein said additive, recessive or dominant genetic model has been selected based on the analysis of a reference (or discovery or training) population of samples (see Examples).

The obtained genetic score is then compared to one or more threshold (or cut-off) values in order to define an autism risk level.

Depending on the number of threshold values, two or more categories of subjects will be defined. Preferably, the number of threshold values is comprised between 1 and 4. In particular, 1, 2, 3, or 4 threshold values may be used.

For one threshold value, two categories of subjects will be defined:
Below the threshold value, a category of subjects with a lower risk of autism than the prevalence of autism in the reference population of subjects,
Above the threshold value, a category of subjects with a higher risk of autism than the prevalence of autism in the reference population of subjects.

By “reference population of subjects” it is meant either the general population (including any individual) or the population of subjects having a sibling with an autism spectrum disorder. The reference population will be selected depending on the nature of the tested subject. If the tested subject is not related to anyone with an autism-spectrum disorder, then the reference population will be the general population (including any individual), in which the prevalence of autism is about 1 per 110 children (i.e. 9.1%). Alternatively, if the tested subject is a sibling of an individual with an autism spectrum disorder, then the reference population will be the population of subjects having a sibling with an autism spectrum disorder, in which the prevalence of autism is about 19%.

The selection of an appropriate threshold value is made based on analysis of a reference (or discovery or training) population of samples, and depending on which feature(s) of the test (specificity, sensitivity, positive predictive value, negative predictive value) is/are considered as the most important. Indeed, features of a test based on a quantitative genetic score can be altered by changing the threshold or cut-off value. Lowering the threshold improves the sensitivity of the test but at the price of lower specificity and more false-positive results. Inversely, raising the cut-off improves the specificity at the price of lower sensitivity and more false negative results.

A multi-risk class test may be constructed using more than one threshold value:
Two threshold values (V1 and V2) may be set to create 3 classes of risk: a reference class (V1≦GS<V2) where the risk is close or equal to the prevalence of the disease in the reference population of subjects, a low risk class (GS<V1) where the risk is lower than the risk in the reference class, and a high risk class (GS≧V2) where the risk is higher than in the reference class.
Three threshold values (V1, V2 and V3) may be set to create 4 classes of risk: a high risk class (V2≦GS<V3), where the risk is higher than the prevalence of the disease in the reference population of subjects; a very high risk class (GS≧V3) where the risk is much higher than the prevalence of the disease in the reference population of subjects; a low risk class (V1≦GS<V2) were the risk is lower than the prevalence of the disease in the reference population of subjects; and a very low risk class (GS<V1) were the risk is much lower than the prevalence of the disease in the reference population of subjects.
Four threshold values (V1, V2, V3 and V4) may be set to create 5 classes of risk: a reference class (V2≦GS<V3) where the risk is close to the prevalence of the disease in the reference population of subjects; a high risk class (V3≦GS<V4), where the risk is higher than in the reference class; a very high risk class (GS≧V4) where the risk is much higher than in the reference class; a low risk class (V1≦GS<V2) were the risk is lower than the risk in the reference class; and a very low risk class (GS<V1) were the risk is much lower than the risk in the reference class.

The number and the value of the different threshold values are settled according to the performance and characteristics expected for the test defined by risk in classes, sensitivity and specificity. Practical examples of determination of one or several appropriate threshold value(s) are described in the experimental section.

Alternatively, a diagnosis of risk of autism, or of a predisposition to autism or of the presence of autism may generally be made if all genotyped SNPs include at least one risk-associated allele. If an additive default genetic model is selected, this corresponds to a genetic score of at least half the maximum genetic score.

For instance, when the genotyped SNPs are rs893109, rs260969, rs12925135, rs2663327, rs636624, rs2367910, rs10787637, rs695083, rs10802802, rs712886, rs12275631, rs3928471 and rs298542, then the subject has a risk of or is predisposed to or has autism when at least one allele of rs893109 is G, at least one allele of rs260969 is T, at least one allele of rs12925135 is C, at least one allele of rs2663327 is C, at least one allele of rs636624 is A, at least one allele of rs2367910 is C, at least one allele of rs10787637 is G, at least one allele of rs695083 is T, at least one allele of rs10802802 is G, at least one allele of rs712886 is C, at least one allele of rs12275631 is T, at least one allele of rs3928471 is T and at least one allele of rs298542 is C.

Similarly, when the genotyped SNPs further include rs12514116, rs16916456, rs7172184, rs8123323, rs10766739, rs16868972, rs12535987, rs2076683, rs9837484, rs314253, rs1485677, rs10150121, rs7534723, rs9370809, rs9940922, rs6063144, rs4782109 and rs2820100, then the subject has or is predisposed to autism when, in addition to the above, at least one allele of rs12514116 is C, at least one allele of rs16916456 is C, at least one allele of rs7172184 is C, at least one allele of rs8123323 is T, at least one allele of rs10766739 is A, at least one allele of rs16868972 is T, at least one allele of rs12535987 is C, at least one allele of rs2076683 is T, at least one allele of rs9837484 is G, at least one allele of rs314253 is G, at least one allele of rs1485677 is G, at least one allele of rs10150121 is C, at least one allele of rs7534723 is A, at least one allele of rs9370809 is T, at least one allele of rs9940922 is G, at least one allele of rs6063144 is C, at least one allele of rs4782109 is T and at least one allele of rs2820100 is C.

Similarly, when the genotyped SNPs further include rs723811, rs11139294, rs11942354, rs1597611, rs4404561, rs6962352, rs7075400, rs35284 and rs946630, then the subject has or is predisposed to autism when, in addition to the above, at least one allele of rs723811 is T, at least one allele of rs11139294 is G, at least one allele of rs11942354 is A, at least one allele of rs1597611 is G, at least one allele of rs4404561 is C, at least one allele of rs6962352 is G, at least one allele of rs7075400 is T, at least one allele of rs35284 is A and at least one allele of rs946630 is G.

Similarly, when the genotyped SNPs further include rs1041644, rs12985015, rs1569284, rs2077642, rs2382104, rs2695112, rs6574041, rs7021928, rs7225320, rs9307866, rs957910, rs6923644, rs1556060, rs1432443, rs220836, rs7974275 and rs10500866, then the subject has or is predisposed to autism when, in addition to the above, at least one allele of rs1041644 is C, at least one allele of rs12985015 is G, at least one allele of rs1569284 is G, at least one allele of rs2077642 is T, at least one allele of rs2382104 is G, at least one allele of rs2695112 is A, at least one allele of rs6574041 is A, at least one allele of rs7021928 is A, at least one allele of rs7225320 is T, at least one allele of rs9307866 is T, at least one allele of rs957910 is T, at least one allele of rs6923644 is G, at least one allele of rs1556060 is G, at least one allele of rs1432443 is T, at least one allele of rs220836 is G, at least one allele of rs7974275 is G and at least one allele of rs10500866 is T.

However, a risk of autism, a predisposition to or the presence of autism in the tested subject is preferably determined based on calculation of a genetic score (GS) and comparison of the GS to one or more threshold values, as described above.

### Linkage Disequilibrium (LD)

Once a first SNP has been identified in a genomic region of interest, the practitioner of ordinary skill in the art can easily identify additional SNPs in linkage disequilibrium with this first SNP. In the context of the invention, the additional SNPs in linkage disequilibrium with a first SNP are within the same gene of said first SNP.

Linkage disequilibrium (LD) is defined as the non-random association of alleles at different loci across the genome. Alleles at two or more loci are in LD if their combination occurs more or less frequently than expected by chance in the population.

For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict.

When there is a causal locus in a DNA region, due to LD, one or more SNPs nearby are likely associated with the trait too. Therefore, any SNPs in LD with a first SNP associated with autism or an associated disorder will be associated with this trait. Identification of additional SNPs in linkage disequilibrium with a given SNP involves: (a) amplifying a fragment from the gene comprising a first SNP from a plurality of individuals; (b) identifying of second SNPs in the gene comprising said first SNP; (c) conducting a linkage disequilibrium analysis between said first SNP and second SNPs; and (d) selecting said second SNPs as being in linkage disequilibrium with said first marker. Subcombinations comprising steps (b) and (c) are also contemplated.

Methods to identify SNPs and to conduct linkage disequilibrium analysis can be carried out by the skilled person without undue experimentation by using well-known methods.

Thus, the practitioner of ordinary skill in the art can easily identify SNPs or combination of SNPs within haplotypes in linkage disequilibrium with the at risk SNP.

Such markers are mapped and listed in public databases like HapMap as well known to the skilled person. Genomic LD maps have been generated across the genome, and such LD maps have been proposed to serve as framework for mapping disease-genes (Risch et al, 1996; Maniatis et al, 2002; Reich et al, 2001). If all polymorphisms in the genome were independent at the population level (i.e., no LD), then every single one of them would need to be investigated in association studies, to assess all the different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

The two metrics most commonly used to measure LD are D′ and r² and can be written in terms of each other and allele frequencies. Both measures range from 0 (the two alleles are independent or in equilibrium) to 1 (the two allele are completely dependent or in complete disequilibrium), but with different interpretation. |D′| is equal to 1 if at most two or three of the possible haplotypes defined by two markers are present, and <1 if all four possible haplotypes are present. r² measures the statistical correlation between two markers and is equal to 1 if only two haplotypes are present.

Most SNPs in humans probably arose by single base modifying events that took place within chromosomes many times ago. A single newly created allele, at its time of origin, would have been surrounded by a series of alleles at other polymorphic loci like SNPs establishing a unique grouping of alleles (i.e. haplotype). If this specific haplotype is transmitted intact to next generations, complete LD exists between the new allele and each of the nearby polymorphisms meaning that these alleles would be 100% predictive of the new allele. Thus, because of complete LD (D′=1 or r²=1) an allele of one polymorphic marker can be used as a surrogate for a specific allele of another. Event like recombination may decrease LD between markers. But, moderate (i.e. 0.5≦r²<0.8) to high (i.e. 0.8≦r²<1) LD conserve the “surrogate” properties of markers. In LD based association studies, when LD exist between markers and an unknown pathogenic allele, then all markers show a similar association with the disease. In a study by Philippi et al (2007), a set of SNPs in strong LD has been shown to be significantly associated to autism (Table 3 for association results and FIG. 2 for LD plots in Philippi et al. (2007)) demonstrating that a set of 5 SNPs (rs1131611, rs11959298, rs6872664, rs6596188 and rs6596189) could be used as surrogate markers for an unknown pathogenic allele in LD with the 5 SNPs. Similar results were observed for different association studies in autism: for two SNPs in high LD within EN2 gene (r2>0.8 for rs1861972 and rs1861973 in Gharani et al (2004)), ASMT gene (D′=0.94 for rs4446909 and 5989681 in Melke et al. (2008)) or NRCAM (four SNPs with D′ between 0.64 and 1 in Marui et al. (2008)). Alternatively, if one SNP did not provide association to the disease, SNPs in high or moderate LD will not provide association: among four SNPs flanking SP1 genes in high LD (r2 between 0.77 and 0.91) and 4 SNPs flanking SUB1 gene (r2 between 0.79 and 0.95), none displayed any association to autism (Campbell et al. (2008)) suggesting an absence of pathogenic variant in LD with the SNPs.

It is well known that many SNPs have alleles that show strong LD (or high LD, defined as r²≧0.80) with other nearby SNP alleles and in regions of the genome with strong LD, a selection of evenly spaced SNPs, or those chosen on the basis of their LD with other SNPs (proxy SNPs or Tag SNPs), can capture most of the genetic information of SNPs, which are not genotyped with only slight loss of statistical power. In association studies, this region of LD are adequately covered using few SNPs (Tag SNPs) and a statistical association between a SNP and the phenotype under study means that the SNP is a causal variant or is in LD with a causal variant. It is a general consensus that a proxy (or Tag SNP) is defined as a SNP in LD (r²≧0.8) with one or more other SNPs. The genotype of the proxy SNP could predict the genotype of the other SNP via LD and inversely. In particular, any SNP in LD with one of the SNPs used herein may be replaced by one or more proxy SNPs defined according to their LD as r²≧0.8.

These SNPs in linkage disequilibrium can also be used in the methods according to the present invention, and more particularly in the diagnostic methods according to the present invention. In particular, the presence of SNPs in linkage disequilibrium (LD) with the above identified SNPs may be genotyped, in place of, or in addition to, said identified SNPs. In the context of the present invention, the SNPs in linkage disequilibrium with the above identified SNP are within the same gene of the above identified SNP. Therefore, in the present invention, the presence of SNPs in linkage disequilibrium (LD) with a SNP of interest and located within the same gene as the SNP of interest may be genotyped, in place of, or in addition to, said SNP of interest. Preferably, such an SNP and the SNP of interest have r²≧0.70, preferably r²≧0.75, more preferably r²≧0.80, and/or have D′≧0.60, preferably D′≧0.65, D′≧0.7, D′≧0.75, more preferably D′≧0.80. Most preferably, such an SNP and the SNP of interest have r²≧0.80, which is used as reference value to define “LD” between SNPs.

### Gender Specificity

The invention further provides a method of determining a risk of autism, or of detecting the predisposition or presence of autism in a male subject, the method comprising genotyping a SNP in the gene loci of at least HTR5A, MACF1, RBFOX1, ABR, PTPRG, and CACNA2D1, in a sample from said subject. Preferably, the SNP in HTR5A is rs893109 (position 27 of SEQ ID NO: 31), in MACF1 is rs260969 (position 27 of SEQ ID NO: 15), in RBFOX1 is rs12925135 (position 27 of SEQ ID NO: 39), in ABR is rs2663327 (position 27 of SEQ ID NO: 40), in PTPRG is rs636624 (position 27 of SEQ ID NO: 22), and/or in CACNA2D1 is rs2367910 (position 27 of SEQ ID NO: 41). Most preferably, all SNPs genotyped are those mentioned in previous sentence. Therefore, in male subjects, the method more particularly comprises genotyping at least rs893109, rs260969, rs12925135, rs2663327, rs636624 and rs2367910.

Preferably, the method further comprises genotyping a SNP in the gene loci of any or all of KCNIP1, UGCG, NTRK3, PLCB1, NELL1, GPR98, MAGI2, and PLAGL1, or combinations thereof. In this case, advantageously, the SNP in KCNIP1 is rs12514116 (position 27 of SEQ ID NO: 38), in UGCG is rs16916456 (position 27 of SEQ ID NO: 11), in NTRK3 is rs7172184 (position 27 of SEQ ID NO: 28), in PLCB1 is rs8123323 (position 27 of SEQ ID NO: 37), in NELL1 is rs10766739 (position 27 of SEQ ID NO: 3), in GPR98 is rs16868972 (position 27 of SEQ ID NO: 42), in MAGI2 is rs12535987 (position 27 of SEQ ID NO: 43), and/or in PLAGL1 is rs2076683 (position 27 of SEQ ID NO: 12). Therefore, preferably, in male subjects, the method further comprises genotyping any or all of the following SNPs rs12514116, rs16916456, rs7172184, rs8123323, rs10766739, rs16868972, rs12535987 and rs2076683, or combinations thereof.

More preferably, the method further comprises genotyping a SNP in the gene loci of any or all of NRG1, TRIM2, EPHA5, PCDH10, and HIP1, or combinations thereof. In this case, advantageously, the SNP in NRG1 is rs723811 (position 27 of SEQ ID NO: 44), in TRIM2 is rs11942354 (position 27 of SEQ ID NO: 45), in EPHA5 is rs1597611 (position 27 of SEQ ID NO: 10), in PCDH10 is rs4404561 (position 27 of SEQ ID NO: 20), and/or in HIP1 is rs6962352 (position 27 of SEQ ID NO: 25). Most preferably, all SNPs genotyped are those mentioned in previous sentence. Therefore, more preferably, in male subjects, the method further comprises genotyping any or all of the following SNPs rs723811, rs11942354, rs1597611, rs4404561 and rs6962352, or combinations thereof.

Even more preferably, the method further comprises genotyping a SNP in the gene loci of any or all of PDE11A, AKAP7, DCLK1, KCNH5, GRIN2A, ACCN1, DCLK2, ASTN2, GRM7, MAP2K1, CADM1, and GRIN2B, or combinations thereof. In this case, advantageously, the SNP in PDE11A is rs2695112 (position 27 of SEQ ID NO: 16), in AKAP7 is rs6923644 (position 27 of SEQ ID NO: 46), near 3′ of DCLK1 is rs1556060 (position 27 of SEQ ID NO: 47), in KCNH5 is rs1041644 (position 27 of SEQ ID NO: 2), in GRIN2A is rs4782109 (position 27 of SEQ ID NO: 21), in ACCN1 is rs7225320 (position 27 of SEQ ID NO: 29), in DCLK2 is rs9307866 (position 27 of SEQ ID NO: 32), in ASTN2 is rs7021928 (position 27 of SEQ ID NO: 26), in GRM7 is rs1569284 (position 27 of SEQ ID NO: 9), in MAP2K1 is rs1432443 (position 27 of SEQ ID NO: 48), in CADM1 is rs220836 (position 27 of SEQ ID NO: 49), and/or in GRIN2B is rs7974275 (position 27 of SEQ ID NO: 50). Most preferably, all SNPs genotyped are those mentioned in previous sentence. Thus, the method preferably further comprises genotyping any or all of the SNP selected from the group of rs2695112, rs6923644, rs1556060, rs1041644, rs4782109, rs7225320, rs9307866, rs7021928, rs1569284, rs1432443, rs220836, and rs7974275, or combinations thereof. In a preferred embodiment, the invention further provides a method of determining a risk of autism, or of detecting the predisposition or presence of autism in a male subject, the method comprising genotyping any SNP as identified in Table 1 or in Table 5.

Table 5 (see below) describes the SNPs useful for the detection of autism in males according to their degree of reproducibility. Their AUCs (Area Under Curves) and associated p-value are also provided.

In these methods, detecting the combined presence of risk-associated alleles, preferably as defined in Table 1, is indicative of a risk of autism, a predisposition to autism, or presence of autism in the male subject. More particularly, the autism risk level is determined as described above, by combining the risk-associated SNPs into a genetic score and comparing it to one or more threshold values. In particular, the combined presence of a G for rs893109, a T for r rs260969, a C for rs12925135, a C for rs2663327, a A for rs636624 and a C for rs2367910 is indicative of a subject being at risk with, predisposed to, or having autism (A genetic score built from these 6 SNPs as described in the Example section is associated to a RI≧0.95, an AUC of 0.64 with p=5.5×10⁻⁸).

The method may also further comprise genotyping a SNP in the gene loci of any or all of PITX1, ATP2B2, EN2, JARID2, CNTNAP2, and HOXA1, or combinations thereof, preferably the method further comprises genotyping any or all of the SNP selected from the group consisting rs6872664, rs2278556, rs1861972, rs7766973, rs7794745, and rs10951154, or combinations thereof.

The invention further provides a method of determining a risk of autism, or of detecting the predisposition or presence of autism in a female subject, the method comprising genotyping a SNP in the gene loci of at least CHRM3, DSCAML1, PTPRG, GFRA1, LPPR4, DLG2, SLC9A9 and BASP1, in a sample from said subject. Preferably, the SNP in CHRM3 is rs10802802 (position 27 of SEQ ID NO: 5), in DSCAML1 is rs695083 (position 27 of SEQ ID NO: 24), in PTPRG is rs636624 (position 27 of SEQ ID NO: 22), in LPPR4 is rs712886 (position 27 of SEQ ID NO: 27), in DLG2 is rs12275631 (position 27 of SEQ ID NO: 51), in SLC9A9 is rs3928471 (position 27 of SEQ ID NO: 19), in BASP1 is rs298542 (position 27 of SEQ ID NO: 52). Most preferably, all SNPs genotyped are those mentioned in previous sentence. Therefore, in female subjects, the method more particularly comprises genotyping at least rs10787637, rs636624, rs695083, rs10802802, rs712886, rs12275631, rs3928471 and rs298542.

Preferably, the method further comprises genotyping a SNP in the gene loci of any or all of CNTN6, NTRK3, DLG4, ERC2, TRIM9, SYT14, JARID2, CDH13, SULF2, GRIN2A and NRG3, or combinations thereof. In this case, advantageously, the SNP in CNTN6 is rs9837484 (position 27 of SEQ ID NO: 35), in NTRK3 is rs7172184 (position 27 of SEQ ID NO: 28), in DLG4 is rs314253 (position 27 of SEQ ID NO: 17), in ERC2 is rs1485677 (position 27 of SEQ ID NO: 8), in TRIM9 is rs10150121 (position 27 of SEQ ID NO: 1), in SYT14 is rs7534723 (position 27 of SEQ ID NO: 30), in JARID2 is rs9370809 (position 27 of SEQ ID NO: 33), in CDH13 is rs9940922 (position 27 of SEQ ID NO: 36), in SULF2 is rs6063144 (position 27 of SEQ ID NO: 53), in GRIN2A is rs4782109 (position 27 of SEQ ID NO: 21), and/or in NRG3 is rs2820100 (position 27 of SEQ ID NO: 54) or rs7075400 (position 27 of SEQ ID NO: 55). Most preferably, all SNPs genotyped are those mentioned in previous sentence. Thus, preferably, in female subjects, the method further comprises genotyping any or all of the following SNPs rs9837484, rs7172184, rs314253, rs1485677, rs10150121, rs7534723, rs9370809, rs9940922, rs6063144, rs4782109 and rs2820100, or combinations thereof.

More preferably, the method further comprises genotyping a SNP in the gene loci of any or all of APBA1, ABR, NRG3, PDE4D and EGLN3, or combinations thereof. In this case, advantageously, the SNP in APBA1 is rs11139294 (position 27 of SEQ ID NO: 6), in ABR is rs2663327 (position 27 of SEQ ID NO: 40), in NRG3 is rs7075400 (position 27 of SEQ ID NO: 55), in PDE4D is rs35284 (position 27 of SEQ ID NO: 18), and/or in EGLN3 is rs946630 (position 27 of SEQ ID NO: 56). Most preferably, all SNPs genotyped are those mentioned in previous sentence. Thus, more preferably, in female subjects, the method further comprises genotyping any or all of the following SNPs rs11139294, rs2663327, rs7075400, rs35284 and rs946630, or combinations thereof.

Even more preferably, the method further comprises genotyping a SNP in the gene loci of any or all of RGS6, SLC24A2, PTPRD, NAV2, PCDH10, MAP1S, and PAX2, or combinations thereof. In this case, advantageously, the SNP in RGS6 is rs6574041 (position 27 of SEQ ID NO: 23), in SLC24A2 is rs957910 (position 27 of SEQ ID NO: 34), in PTPRD is rs2382104 (position 27 of SEQ ID NO: 14), in NAV2 is rs10500866 (position 27 of SEQ ID NO: 57), in PCDH10 is rs4404561 (position 27 of SEQ ID NO: 20), in MAP1S is rs12985015 (position 27 of SEQ ID NO: 7), and/or in PAX2 is rs2077642 (position 27 of SEQ ID NO: 13). Most preferably, all SNPs genotyped are those mentioned in previous sentence. Thus, the method preferably further comprises genotyping any or all of the SNP selected from the group consisting rs6574041, rs957910, rs2382104, rs10500866, rs4404561, rs12985015, and rs2077642, or combinations thereof. In a preferred embodiment, the invention further provides a method of determining a risk of autism, or of detecting the predisposition or presence of autism in a female subject, the method comprising genotyping any SNP as identified in Table 1 or in Table 6.

Table 6 (see below) describes the SNPs useful for the detection of autism in females according to their degree of reproducibility. Their AUCs and associated p-value are also provided.

In these methods, detecting the combined presence of risk-associated alleles, preferably as defined in Table 1, is indicative of a risk of autism, a predisposition to autism, or presence of autism in the female subject. More particularly, the autism risk level is determined as described above, by combining the risk-associated SNPs into a genetic score and comparing it to one or more threshold values. In particular, the combined presence of a G for rs10802802, a T for rs695083, a A for rs636624, a G for rs10787637, a C for rs712886, a T for rs12275631, a T for rs3928471 and a C for rs298542 is indicative of a subject being at risk with, predisposed to, or having autism (A genetic score built from these 8 SNPs as described in the Example section is associated to a RI≧0.95 an AUC of 0.69 with p=1.77×10⁻⁹).

The method may also further comprise genotyping a SNP in the gene loci of any or all of EN2, JARID2, MARK1, ITGB3, and CNTNAP2, or combinations thereof, preferably the method further comprises genotyping any or all of the SNP selected from the group consisting rs1861972, rs7766973, rs12410279, rs5918, and rs7794745, or combinations thereof.

TABLE 5
AUCs and associated Pvalue for genetic score built
from SNPs with different degree of reproducibility
in the Discovery and Validation sample in males.

| RI | RI ≧ 0.95 | RI ≧ 0.90 | RI ≧ 0.85 | RI ≧ 0.80 |
|---|---|---|---|---|
| definition | (6 SNPs) | (14 SNPs) | (19 SNPs) | (31 SNPs) |
| AUC | 0.64 | 0.68 | 0.70 | 0.7 |
| Pvalue | 5.5 × 10⁻⁸ | 3.04 × 10⁻⁸ | 6.01 × 10⁻¹³ | 2.6 × 10⁻¹² |
| SNP list | rs893109 | rs893109 | rs893109 | rs893109 |
| | rs260969 | rs12514116 | rs12514116 | rs12514116 |
| | rs12925135 | rs16916456 | rs16916456 | rs16916456 |
| | rs2663327 | rs260969 | rs260969 | rs260969 |
| | rs636624 | rs12925135 | rs12925135 | rs12925135 |
| | rs2367910 | rs2663327 | rs2663327 | rs2663327 |
| | | rs636624 | rs636624 | rs636624 |
| | | rs7172184 | rs7172184 | rs7172184 |
| | | rs2367910 | rs2367910 | rs2367910 |
| | | rs8123323 | rs8123323 | rs8123323 |
| | | rs10766739 | rs10766739 | rs10766739 |
| | | rs16868972 | rs16868972 | rs16868972 |
| | | rs12535987 | rs12535987 | rs12535987 |
| | | rs2076683 | rs2076683 | rs2076683 |
| | | | rs723811 | rs723811 |
| | | | rs11942354 | rs11942354 |
| | | | rs1597611 | rs1597611 |
| | | | rs4404561 | rs4404561 |
| | | | rs6962352 | rs6962352 |
| | | | | rs2695112 |
| | | | | rs6923644 |
| | | | | rs1556060 |
| | | | | rs1041644 |
| | | | | rs4782109 |
| | | | | rs7225320 |
| | | | | rs9307866 |
| | | | | rs7021928 |
| | | | | rs1569284 |
| | | | | rs1432443 |
| | | | | rs220836 |
| | | | | rs7974275 |

TABLE 6
AUCs and associated Pvalue for genetic score built
from SNPs with different degree of reproducibility
in the Discovery and Validation sample in females

| RI | RI ≧ 0.95 | RI ≧ 0.90 | RI ≧ 0.85 | RI ≧ 0.80 |
|---|---|---|---|---|
| definition | (8 SNPs) | (19 SNPs) | (24 SNPs) | (31 SNPs) |
| AUC | 0.69 | 0.74 | 0.74 | 0.73 |
| Pvalue | 1.77 × 10⁻⁹ | 8.49 × 10⁻¹² | 10⁻¹³ | 2.7 × 10⁻¹² |
| SNP list | rs10787637 | rs10787637 | rs10787637 | rs10787637 |
| | rs636624 | rs636624 | rs636624 | rs636624 |
| | rs695083 | rs695083 | rs695083 | rs695083 |
| | rs10802802 | rs10802802 | rs10802802 | rs10802802 |
| | rs712886 | rs712886 | rs712886 | rs712886 |
| | rs12275631 | rs12275631 | rs12275631 | rs12275631 |
| | rs3928471 | rs3928471 | rs3928471 | rs3928471 |
| | rs298542 | rs298542 | rs298542 | rs298542 |
| | | rs9837484 | rs9837484 | rs9837484 |
| | | rs7172184 | rs7172184 | rs7172184 |
| | | rs314253 | rs314253 | rs314253 |
| | | rs1485677 | rs1485677 | rs1485677 |
| | | rs10150121 | rs10150121 | rs10150121 |
| | | rs7534723 | rs7534723 | rs7534723 |
| | | rs9370809 | rs9370809 | rs9370809 |
| | | rs9940922 | rs9940922 | rs9940922 |
| | | rs6063144 | rs6063144 | rs6063144 |
| | | rs4782109 | rs4782109 | rs4782109 |
| | | rs2820100 | rs2820100 | rs2820100 |
| | | | rs11139294 | rs11139294 |
| | | | rs2663327 | rs2663327 |
| | | | rs7075400 | rs7075400 |
| | | | rs35284 | rs35284 |
| | | | rs946630 | rs946630 |
| | | | | rs6574041 |
| | | | | rs957910 |
| | | | | rs2382104 |
| | | | | rs10500866 |
| | | | | rs4404561 |
| | | | | rs12985015 |
| | | | | rs2077642 |

### Genotyping Methods and Kits

The term “genotyping” means determining the allele of the recited SNPs, which allows detecting the presence of a autism risk-associated allele.

The SNP in the gene locus may be genotyped by sequencing, selective hybridisation and/or selective amplification.

Sequencing can be carried out using techniques well known in the art, using automatic sequencers. The sequencing may be performed on the complete genes or, more preferably, on specific domains thereof, typically those known or suspected to carry deleterious mutations or other alterations.

Amplification is based on the formation of specific hybrids between complementary nucleic acid sequences that serve to initiate nucleic acid reproduction.

Amplification may be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Amplification usually requires the use of specific nucleic acid primers, to initiate the reaction.

Nucleic acid primers useful for amplifying sequences from the gene or locus are able to specifically hybridize with a portion of the gene locus that flanks a target region of said locus, said target region being altered in certain subjects having autism.

Preferred technique uses allele-specific PCR (AS-PCR). This technique allows amplification to target specific alleles. AS-PCR is performed with three primers including two primers with the same nucleotide sequences except in 3′ direction with one base corresponding to the specific allele. Additionally, two universal primers which are coupled to a specific fluorophore are used. These two primers will transmit a signal if they are incorporated in a PCR product (Nazarenko et al. 1997; Myakishev et al. 2001).

This technique can be performed in a single tube, in a microplate and run in a classical qPCR system. But the new platforms of micro-fluidic can also be used for running this technique, with the advantage to interrogate in parallel several ten of samples on several ten of markers.

As an example: The Fluidigm Dynamic Array as large as a 96-well plate allows a study of 96 SNP on 96 samples; therefore 9216 reactions of PCR are performed in parallel. The samples and primers are distributed in reaction chambers of a few nanoliters by a system of micro-fluidics.

Fluidigm Dynamic Array integrated fluidic circuits (IFCs) have an on-chip network of microfluidic channels, chambers and valves that automatically assemble individual PCR reactions, decreasing the number of pipetting steps required by up to 100 fold. After loading the samples and primers onto the Dynamic Arrays, the PCR is then performed on BioMark or EP1 System integrating thermal cycling and fluorescences detection on Integrated fluidic circuits. (Wang et al 2009(b))

Hybridization detection methods are based on the formation of specific hybrids between complementary nucleic acid sequences that serve to detect nucleic acid sequence alteration(s).

A particular detection technique involves the use of a nucleic acid probe specific for wild type or altered gene, followed by the detection of the presence of a hybrid. The probe may be in suspension or immobilized on a substrate or support (as in nucleic acid array or chips technologies). The probe is typically labelled to facilitate detection of hybrids.

In a preferred embodiment, an alteration in the gene locus is determined by DNA chip analysis. Such DNA chip or nucleic acid microarray consists of different nucleic acid probes that are chemically attached to a substrate, which can be a microchip, a glass slide or a microsphere-sized bead. A microchip may be constituted of polymers, plastics, resins, polysaccharides, silica or silica-based materials, carbon, metals, inorganic glasses, or nitrocellulose. Probes comprise nucleic acids such as cDNAs or oligonucleotides that may be about 10 to about 60 base pairs. To determine the alteration of the genes, a sample from a test subject is labelled and contacted with the microarray in hybridization conditions, leading to the formation of complexes between target nucleic acids that are complementary to probe sequences attached to the microarray surface. The presence of labelled hybridized complexes is then detected. Many variants of the microarray hybridization technology are available to the man skilled in the art (see e.g. the review by Kidgell&Winzeler, 2005).

The invention further provides a kit comprising primers pairs (forward and reverse primers) or triplets (two forward and one reverse primers) and/or probes for the specific detection of a SNP in the gene loci of at least HTR5A, MACF1, RBFOX1, ABR, PTPRG, CACNA2D1, GFRA1, DSCAML1, CHRM3, LPPR4, DLG2, SLC9A9 and BASP1, preferably the SNPs are rs893109 in HTR5A (position 27 of SEQ ID NO: 31), rs260969 in MACF1 (position 27 on SEQ ID NO: 15), rs12925135 in RBFOX1 (position 27 of SEQ ID NO: 39), rs2663327 in ABR (position 27 of SEQ ID NO: 40), rs636624 in PTPRG (position 27 of SEQ ID NO: 22), rs2367910 in CACNA2D1 (position 27 of SEQ ID NO: 41), rs10787637 in GFRA1 (position 27 of SEQ ID NO: 4), rs695083 in DSCAML1 (position 27 of SEQ ID NO: 24), rs10802802 in CHRM3 (position 27 of SEQ ID NO: 5), rs712886 in LPPR4 (position 27 of SEQ ID NO: 27), rs12275631 in DLG2 (position 27 of SEQ ID NO: 51), rs3928471 in SLC9A9 (position 27 of SEQ ID NO: 19), and rs298542 in BASP1 (position 27 of SEQ ID NO: 52).

The kit may further comprise primers pairs (forward and reverse primers) or triplets (two forward and one reverse primers) and/or probes for the specific detection of a SNP in the gene loci of any or all of KCNIP1, UGCG, NTRK3, PLCB1, NELL1, GPR98, MAGI2, PLAGL1, CNTN6, DLG4, ERC2, TRIM9, SYT14, JARID2, CDH13, SULF2, GRIN2A and NRG3, or combinations thereof, preferably the kit further comprises primers pairs (forward and reverse primers) or triplets (two forward and one reverse primers) and/or probes for the specific detection of any or all of rs12514116 in KCNIP1 (position 27 on SEQ ID NO: 38), rs16916456 in UGCG (position 27 of SEQ ID NO: 11), rs7172184 in NTRK3 (position 27 of SEQ ID NO: 28), rs8123323 in PLCB1 (position 27 of SEQ ID NO: 37), rs10766739 in NELL1 (position 27 of SEQ ID NO: 3), rs16868972 in GPR98 position 27 of SEQ ID NO: 42), rs12535987 in MAGI2 (position 27 of SEQ ID NO: 43), rs207668 in PLAGL1 (position 27 of SEQ ID NO: 12), rs9837484 in CNTN6 (position 27 of SEQ ID NO: 35), rs314253 in DLG4 (position 27 of SEQ ID NO: 17), rs1485677 in ERC2 (position 27 of SEQ ID NO: 8), rs10150121 in TRIM9 (position 27 of SEQ ID NO: 1), rs7534723 in SYT14 (position 27 of SEQ ID NO: 30), rs9370809 in JARID2 (position 27 of SEQ ID NO: 33), rs9940922 in CDH13 (position 27 of SEQ ID NO: 36), rs6063144 in SULF2 (position 27 of SEQ ID NO: 53), rs4782109 in GRIN2A (position 27 of SEQ ID NO: 21), and rs2820100 in NRG3 (position 27 of SEQ ID NO: 54), or combinations thereof.

Said kit may also or in addition further comprises primers pairs (forward and reverse primers) or triplets (two forward and one reverse primers) and/or probes for the specific detection of a SNP in the gene loci of any or all of NRG1, TRIM2, EPHA5, PCDH10, HIP1, APBA1, PDE4D and EGLN3, or combinations thereof, preferably the kit further comprises primers pairs (forward and reverse primers) or triplets (two forward and one reverse primers) and/or probes for the specific detection of any or all of rs723811 in NRG1 (position 27 on SEQ ID NO: 44), rs11139294 in APBA1 (position 27 of SEQ ID NO: 6), rs11942354 in TRIM2 (position 27 of SEQ ID NO: 45), rs1597611 in EPHA5 (position 27 of SEQ ID NO: 10), rs4404561 in PCDH10 (position 27 of SEQ ID NO: 20), rs6962352 in HIP1 (position 27 of SEQ ID NO: 25), rs7075400 in NRG3 (position 27 of SEQ ID NO: 55), rs35284 in PDE4D (position 27 of SEQ ID NO: 18) and rs946630 in EGLN3 (position 27 of SEQ ID NO: 56), or combinations thereof.

Said kit may also or in addition further comprises primers pairs (forward and reverse primers) or triplets (two forward and one reverse primers) and/or probes for the specific detection of at least one SNP in the gene loci selected from the group consisting of ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, or combinations thereof, preferably the kit further comprises primers pairs (forward and reverse primers) or triplets (two forward and one reverse primers) and/or probes for the specific detection of any or all of rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456, or combinations thereof.

In particular, the kit may comprise primers pairs (forward and reverse primers) or triplets (two forward and one reverse primers) and/or probes for the specific detection of at least one SNP in all of the following the gene loci: ABR, ACCN1, AKAP7, APBA1, ASTN2, BASP1, CACNA2D1, CADM1, CDH13, CHRM3, CNTN6, DCLK1, DCLK2, DLG2, DLG4, DSCAML1, EGLN3, EPHA5, ERC2, GFRA1, GPR98, GRIN2A, GRIN2B, GRM7, HIP1, HTR5A, JARID2, KCNH5, KCNIP1, LPPR4, MACF1, MAGI2, MAP1S, MAP2K1, NAV2, NELL1, NRG1, NRG3, NTRK3, PAX2, PCDH10, PDE11A, PDE4D, PLAGL1, PLCB1, PTPRD, PTPRG, RBFOX1, RGS6, SLC24A2, SLC9A9, SULF2, SYT14, TRIM2, TRIM9 and UGCG, preferably the kit comprises primers pairs (forward and reverse primers) or triplets (two forward and one reverse primers) and/or probes for the specific detection of all following SNPs: rs2663327, rs7225320, rs6923644, rs11139294, rs7021928, rs298542, rs2367910, rs220836, rs9940922, rs10802802, rs9837484, rs1556060, rs9307866, rs12275631, rs314253, rs695083, rs946630, rs1597611, rs1485677, rs10787637, rs16868972, rs4782109, rs7974275, rs1569284, rs6962352, rs893109, rs9370809, rs1041644, rs12514116, rs712886, rs260969, rs12535987, rs12985015, rs1432443, rs10500866, rs10766739, rs723811, rs2820100, rs7075400, rs7172184, rs2077642, rs4404561, rs2695112, rs35284, rs2076683, rs8123323, rs2382104, rs636624, rs12925135, rs6574041, rs957910, rs3928471, rs6063144, rs7534723, rs11942354, rs10150121, rs16916456.

The kit may also further comprise primers pairs (forward and reverse primers) or triplets (two forward and one reverse primers) and/or probes for the specific detection of a SNP in the gene loci of any or all of PITX1, ATP2B2, EN2, JARID2, MARK1, ITGB3, CNTNAP2, and HOXA1, or combinations thereof, preferably the kit further comprises primers pairs (forward and reverse primers) or triplets (two forward and one reverse primers) and/or probes for the specific detection of any or all of the SNP selected from the group consisting rs6872664, rs2278556, rs1861972, rs7766973, rs12410279, rs5918, rs7794745, and rs10951154, or combinations thereof. These genes/SNPs are those described in Carayol et al, 2011 and WO2011/138372.

Primer pairs (forward and reverse primers) or triplets (two forward and one reverse primers) may be used for specific amplification of part of a target gene comprising the SNP of interest. When only two primers are used, they are generally located each on one side of the target SNP of interest and are used in order to increase the amount of target sequence for further analysis. When three primers are used, the single reverse primer is preferably located on one side of the target SNP of interest, while the two corresponding forward primers are respectively specific of the protective or risk-associated allele of the SNP. The base differing between the two primers is preferably located in 3′ of the forward primers. Primers are polynucleotides of about 15 to about 25 nucleotides, preferably of about 18 to about 22 nucleotides.

A probe for the specific detection of a SNP in a gene locus may notably comprise or consist of a polynucleotide comprising at least 10 contiguous bases, preferably about 10 to about 60 bases, complementary to part of a target gene comprising the SNP of interest.

In particular, the invention provides a set of polynucleotides comprising at least 10 contiguous bases, preferably about 10 to about 60 bases, of (i) SEQ ID NO: 31, 15, 39, 40, 22, 41, 4, 24, 5, 27, 51, 19 and 52 respectively around position 27 of SEQ ID NO: 31, position 27 of SEQ ID NO:15, position 27 of SEQ ID NO: 39, position 27 of SEQ ID NO: 40, position 27 of SEQ ID NO: 22, position 27 of SEQ ID NO: 41, position 27 of SEQ ID NO: 4, position 27 of SEQ ID NO: 24, position 27 of SEQ ID NO:5, position 27 of SEQ ID NO: 27, position 27 of SEQ ID NO: 51 and position 27 of SEQ ID NO: 19, or (ii) of the complement of said sequences. Such a set of polynucleotides may further comprise polynucleotides comprising at least 10 contiguous bases, preferably about 10 to about 60 bases, of (i) SEQ ID NO:38, SEQ ID NO:11, SEQ ID NO:28, SEQ ID NO:37, SEQ ID NO:3, SEQ ID NO:42, SEQ ID NO:43, SEQ ID NO:12, SEQ ID NO:35, SEQ ID NO:17, SEQ ID NO:8, SEQ ID NO:1, SEQ ID NO:30, SEQ ID NO:33, SEQ ID NO:36, SEQ ID NO:53, SEQ ID NO:21, SEQ ID NO:54 and SEQ ID NO:55, respectively around positions of SEQ ID NO:38, SEQ ID NO:11, SEQ ID NO:28, SEQ ID NO:37, SEQ ID NO:3, SEQ ID NO:42, SEQ ID NO:43, SEQ ID NO:12, SEQ ID NO:35, SEQ ID NO:17, SEQ ID NO:8, SEQ ID NO:1, SEQ ID NO:30, SEQ ID NO:33, SEQ ID NO:36, SEQ ID NO:53, SEQ ID NO:21, SEQ ID NO:54 and SEQ ID NO:55 mentioned in Table 1, or (ii) of the complement of said sequences. Such a set of polynucleotides may further comprise polynucleotides comprising at least 10 contiguous bases, preferably about 10 to about 60 bases, of (i) SEQ ID NO:44, SEQ ID NO:45, SEQ ID NO:10, SEQ ID NO:20, SEQ ID NO:25, SEQ ID NO:6, SEQ ID NO:18 and SEQ ID NO:56, respectively around positions of SEQ ID NO:44, SEQ ID NO:45, SEQ ID NO:10, SEQ ID NO:20, SEQ ID NO:25, SEQ ID NO:6, SEQ ID NO:18 and SEQ ID NO:56 mentioned in Table 1, or (ii) of the complement of said sequences.

In a preferred embodiment, the invention provides a set of polynucleotides comprising at least 10 contiguous bases, preferably about 10 to about 60 bases, of (i) each of SEQ ID NO:1 to SEQ ID NO:57, respectively around positions of SEQ ID NO:1 to SEQ ID NO:57 mentioned in Table 1, or (ii) of the complement of said sequences.

The above sets of polynucleotides may further comprise at least 10 contiguous bases, preferably about 10 to about 60 bases, of (i) each of SEQ ID NO:58 to SEQ ID NO:65, respectively around positions of SEQ ID NO:58 to SEQ ID NO:65 mentioned in Table 2, or (ii) of the complement of said sequences.

Preferably, the kit according to the invention is dedicated to the genotyping of the target SNPs of interest. By “dedicated”, it is meant that primer pairs (forward and reverse primers) or triplets (two forward and one reverse primers) and/or probes for the specific detection of a SNP in the kit of the invention essentially consist of those necessary to the specific detection of the SNPs of interest, and thus comprise a minimum of primer pairs (forward and reverse primers) or triplets (two forward and one reverse primers) and/or probes for the specific detection of other SNPs than those mentioned above. For instance, a dedicated kit of the invention preferably comprises no more than 50, 40, 30, 25, 20, preferably no more than 15, no more than 14, no more than 13, no more than 12, no more than 11, preferably no more than 10, preferably no more than 9, 8, 7, 6, 5, 4, 3, 2, or 1 primer pairs (forward and reverse primers) or triplets (two forward and one reverse primers) and/or probes for the specific detection of other SNPs than those mentioned above. The dedicated kit of the invention thus preferably contains no more than 100, 90, 80, preferably no more than 70, no more than 69, no more than 68, no more than 67, no more than 66, preferably no more than 65 distinct primer pairs (forward and reverse primers) or triplets (two forward and one reverse primers) and/or probes for the specific detection of SNPs. It may however contain additional reagents such as a polymerase, buffers or any other useful reagent. It may further contain instructions for determining a risk of autism, a predisposition to autism or the presence of autism. For instance, it may contain instructions for calculating a genetic score and appropriate threshold value(s).

A further subject of the invention is a microarray comprising a set of polynucleotides and optionally, a substrate on which the set of polynucleotides is immobilized, wherein the set of polynucleotides is as defined above. Such a microarray is also preferably dedicated the genotyping of the target SNPs of interest. For a microarray, this means that the specific probes of the microarray essentially consist of probes specific for the target SNPs of interest and only comprise a minimum of probes specific for other SNPs. Preferably, a dedicated microarray comprises no more than 50, 40, 30, 25, 20, preferably no more than 15, no more than 14, no more than 13, no more than 12, no more than 11, preferably no more than 10, preferably no more than 9, 8, 7, 6, 5, 4, 3, 2, or 1 probes for the specific detection of other SNPs than those mentioned above. The dedicated microarray of the invention thus preferably contains no more than 100, 90, 80, preferably no more than 70, no more than 69, no more than 68, no more than 67, no more than 66, preferably no more than 65 distinct probes for the specific detection of SNPs.

Preferably the polynucleotides are immobilized on a substrate coated with an active group selected from the group consisting of amino-silane, poly-L-lysine and aldehyde.

In a particular embodiment, the substrate is composed of a material selected from the group consisting of silicon, glass, quartz, metal and plastic.

### Methods of Treatment

The present invention further relates to methods for treating or preventing autism in a subject, the method comprising:
a) determining a risk of autism, or detecting predisposition to or the presence of autism in a subject by any method according to the invention described herein, and
b) if said subject is determined to be at risk of autism, as predisposed to autism or as suffering from autism, then submitting said subject to:
   i) a behavioral autism instrument, such as Autism Diagnostic Observation Schedule-Generic [ADOS-G],
   ii) an indirect, interview-based autism instrument with third parties, such as Autism Diagnostic Interview-Revised [ADI-R], and/or
   iii) Early Intensive Behavioural Intervention (EIBI).

Preferably, if the subject is determined to be at risk of autism, as predisposed to autism or as suffering from autism, then said subject is first rapidly submitted to a clinical evaluation, including behavioral or an indirect, interview-based autism instrument, preferably the Autism Diagnostic Interview-Revised [ADI-R] test in order to confirm the diagnosis of autism. If autism diagnosis is confirmed, then the subject is rapidly submitted to Early Intensive Behavioural Intervention (EIBI), since early intervention has been found to improve outcome for autistic subjects.

The methods of determining a risk of autism, or of detecting the predisposition to or the presence of autism in a subject according to the invention are mainly intended for screening young and very young children for autism, in particular young brothers or sisters of a child already diagnosed as suffering from autism, as early as possible, even before behavioral autism tests (e.g. Autism Diagnostic Observation Schedule-Generic [ADOS-G]) or indirect, interview-based autism tests with third parties (e.g., Autism Diagnostic Interview-Revised [ADI-R]) may be performed. This may permit to perform such tests and confirm autism as early as possible, thus allowing early therapeutic intervention.

Indeed, the American Academy of Pediatrics (AAP) has published clinical practice guidelines on the early identification, screening and diagnosis of ASD with recommendations that all 18- and 24-months olds be screened for ASD (Johnson and Myers 2007).

If the screening result is positive, the pediatrician should provide peer reviewed and/or consensus-developed ASD materials. Because a positive screening result does not determine a diagnosis of ASD, the child should be referred for a comprehensive ASD evaluation, to early intervention/early childhood education services, and an audiologic evaluation (Johnson and Myers 2007).

There is some evidence that Early Intensive Behavioural Intervention (EIBI)—incorporating the principles of applied behavior analysis (ABA)—is an effective intervention approaches for young children with autism (Dawson and Osterling 1997; Warren et al. 2011; Reichow et al. 2012). However, the current state of the evidence is limited due to the lack of randomized controlled trials.

The only comprehensive EIBI program available for children aged less than 30 months that has been empirically evaluated is the Early Start Denver Model (ESDM) (Dawson et al. 2010). After 2 years of intensive intervention, compared with children who received community-intervention, children who received the ESDM displayed significantly improved IQ with an increased of 17.6 points compared with 7.0 points in the comparison group relative to baseline scores. Children in the comparative group showed greater delays in adaptive behavior. Although, children who received ESDM were more likely to experience a change in diagnosis from autism to pervasive developmental disorder not otherwise specified, than the comparison group. Moreover, the authors demonstrated EIBI was associated with normalized patterns of brain activity, which is associated with improvements in social behavior, in young children with autism spectrum disorder (Dawson et al. 2012).

The lifetime per capita incremental societal cost of autism has been evaluated to $2.2 million (Ganz 2007). In a cost-benefit study of EIBI, Jacobsen and al. estimated the net savings to age 55 for a child with PDD who achieves normal functioning is $1.5 million and the net savings for the child who achieves partial effects is roughly $1 million (Jacobson and Mulick 2000).

Therefore, early screening, followed by early confirmative diagnosis and/or intervention could be very helpful to improve the fate of autistic subject and decrease the cost associated to their management.

The test according to the invention may thus be used for early screening, and followed by confirmative diagnosis and/or intervention if a risk of autism, a predisposition to autism or the presence of autism is diagnosed.

In this case, the confirmative diagnosis may be made using behavioral autism diagnosis instruments (e.g. Autism Diagnostic Observation Schedule-Generic [ADOS-G]) (Gotham et al. 2007) or indirect, interview-based autism diagnosis instruments (e.g., Autism Diagnostic Interview-Revised [ADI-R]) (Lord et al, 1994). Such tests are well known to those skilled in the art.

If autism is confirmed, or even before such confirmative diagnosis may be performed, therapeutic intervention may be performed. There are no evidence-based pharmacotherapies to treat the core symptoms associated with ASD but, as mentioned above, there is some evidence that Early Intensive Behavioural Intervention (EIBI)—incorporating the principles of applied behavior analysis (ABA)—is an effective intervention approaches for young children with autism.

Early Intensive Behavioral Intervention (EIBI) is one of the more well-established treatments for ASD. EIBI is a highly structured teaching approach for young children with

ASD (usually less than five years old), that is rooted in principles of applied behavior analysis (ABA). The origins of EIBI are linked to the University of California at Los Angeles Young Autism Project model (also termed the Lovaas model) (see Lovaas 1981 and Lovaas 1987). The core elements of EIBI involve (a) a specific teaching procedure referred to as discrete trial training, (b) the use of a 1:1 adult-to-child ratio in the early stages of the treatment, and (c) implementation in either home or school settings for a range of 20 to 40 hours per week across one to four years of the child's life (see Eikeseth 2009 and Smith 2010). Typically, EIBI is implemented under the supervision of personnel trained in ABA procedures who systematically follow a treatment manual (for example, Lovaas 1981; Maurice 1996) indicating the scope and sequence of tasks to be introduced and taught. A particular example of EIBI is the Early Start Denver Model (ESDM), which is described in Smith et al, 2008.

The following examples illustrate the present invention without limiting its scope.

### Examples

### 1. Materials and Methods

### Subjects and Genotyping

Two independent sets of autism multiplex family samples were used. The discovery population consisted of 545 multiplex families from the AGRE repository (Lajonchere et al, 2010), including 964 affected siblings (773 males and 191 females; 4.1:1 male to female sex ratio) and 317 unaffected siblings (144 males and 173 females). The validation population consisted of 288 multiplex families from a totally independent collection enriched with a complimentary set of 339 families from AGRE. It was composed by 1 000 affected siblings (812 males and 188 females; 4.3:1 male to female sex ratio) and 288 unaffected siblings (141 males and 147 females). Detailed diagnostic criteria for the AGRE data set can be found on the AGRE website (http://www.agre.org/). Only individuals with a “strict” definition of autism according to the Autism Diagnostic Interview Revisited (ADI-R) were selected to improve the power of GWAS by homogenizing the phenotype (Shao et al, 2002 and McCarthy et al, 2008). Members of the AGRE families were genotyped as previously described (Wang et al, 2009). SNPs that failed Hardy Weinberg Equilibrium Test (P<10⁻³) or that have a call rate less than 90% or a minor allele frequency less than 5% were removed. Mendelian transmissions of alleles were checked for every SNP and genotypes that were inconsistent with Mendelian inheritance in one or several families were set to unknown in all the members of the families showing the error. SNPs identified in the discovery population were genotyped in the validation collection as previously described (Carayol et al, 2011).

### Association Studies

GWAS were performed using the Family Based Association Test (FBAT) software (Laird et al, 2000) under additive and recessive/dominant (in both possible orientations: major allele dominant/minor allele dominant) inheritance models. SNPs with a p-value less than 10⁻³ were tested for their ability to discriminate individuals with autism from their unaffected siblings. A “case-sibling control” association analysis was performed and odds ratio were estimated using a Generalized Estimating Equation (GEE) model to account for the non-independence of individuals from the same family (Zeger et al, 1986). The gender was introduced as an adjustment covariate when it was not used as variable of stratification. Markers associated at the nominal threshold (α=0.05) were selected for subsequent analyses.

### SNP Prioritization

To extract association signals from the GWAS and to minimize false positive SNPs, the inventors have developed a scoring method where points were allotted to statistical parameters, genomic characteristics, previous reporting and physiological properties for each selected SNP and its related gene(s).

### Definition of the Genetic Models and Development of Genetic Scores

Replicability of the genetic models was tested for each SNP internally and externally using bootstrap resampling in the discovery and the validation populations by computing a Reproducibility Index (RI) (Carayol et al, 2011 and Carayol et al, 2010 and Ma et al, 2006). Reproducibility Index was computed as previously described (Carayol et al, 2011):

1. Generation of a ‘pseudo-sample’ consisting of 545 families by randomly sampling the 545 families of the discovery population with replacement.
2. For each tested SNP i, odds ratio (OR) associated with the deleterious allele under additive (OR_{Add,i}), recessive (OR_{Rec,i}) and dominant (OR_{DOM,i}) models were estimated.
3. Steps 1 and 2 were repeated a 1 000 times.
4. For each tested SNP i, computation of M_{GM,i} which represents the number of times the deleterious allele maintains its deleterious effect under each genetic model (GM=additive, recessive, dominant), i.e. the number of times OR_{GM,i}>1.00 in the thousand pseudos-amples in males and females separately.
5. Three RIs for each SNP i, one for each genetic model (GM), were calculated as RI_{GM,i}=M_{GM,i}/1 000 in males and females separately.
6. Repetition of steps 1 to 5 using the validation population.

SNPs were included in genetic scores based on their degree of reproducibility. Considering a stringent RI threshold of 90%, a SNP under a specific genetic model was included in the genetic score (GS90%) if the estimated RI of this model was greater than 90% in both the discovery and validation populations. This highly reproducible model is considered to be the “best-fitting model”. In case more than one model fulfilled this criterion, the model with the highest RI estimated in the validation population was selected. Subsequent genetic score models (GS80% to GS0%) were constructed by adding to the previous set of SNPs new genetic markers under their best-fitting genetic model using relaxed RI thresholds from 80% to 0%. Genetic scores of individuals with autism and their unaffected siblings from the validation population were built as the sum of deleterious alleles under their specific genetic model, as previously described (Carayol et al, 2011). GEE model was used to test the association of the genetic scores with autism and a p-value less than 5% indicated a significant association. Areas under the receiver operating characteristic curve (AUCs) which quantify the ability of the genetic scores to discriminate affected from unaffected individuals were estimated using the “ROC” R package (www.bioconductor.org/packages/devel/bioc/html/ROC.html). Empirical 95% confidence intervals (CIs) were determined by bootstrapping 1 000 times the validation sample using each family as a resampling unit. Positive predictive values were estimated as previously described (Carayol et al, 2011) using sensitivity, specificity and sibling recurrence risk of 25.9% in males and 9.6% in females (Ozonoff et al., 2011).

### 2. Results

Four family-based GWAS were performed on the discovery population of multiplex autism families, three on affected individuals with and without gender stratification and one on their unaffected siblings. In total, 900 SNPs were found to be associated with autism (p-value <10⁻³ in family-based GWAS) and to significantly discriminate affected from unaffected siblings (p-value <0.05 in “case-sibling control” analysis). Specifically, 149 and 237 SNPs were identified through the GWAS conducted on autistic males and females, respectively, 156 when all the affected individuals were analyzed, and 358 from the GWAS on unaffected siblings. Prioritization of these 900 SNPs identifies 133 candidate genetic markers of autism.

### Construction of Genetic Scores (GS)

Gender-specific genetic scores (GS) for affected and unaffected siblings were built using SNPs under their best-fitting genetic model selected depending on the RI threshold considered. The ability of the genetic score models to discriminate affected from unaffected individuals, indicated by the AUC, and their association with autism was assessed for the different genetic score models. Genetic scores built using all the identified SNPs (GS0%) were significantly associated with autism in males (P=1.16×10⁻³) and females (P=5.97×10⁻⁵) with AUCs of 0.59 (95% CI: 0.54-0.65) and 0.65 (95% CI: 0.58-0.72), respectively. AUC estimates increased along with genetic score models and reach their maximum, 0.73 (95% CI: 0.69-0.78) in males and 0.74 (95% CI: 0.68-0.80) in females, for GS60% in males and GS40% in females. A slight decrease of the AUCs to 68% (95% CI: 0.63-0.73) was observed from GS60% to the more stringent GS90% in males whereas AUC estimates remained close to 74% in females from GS40% to GS90%.

Reliability of GS80 in Males and Females with Varying Number of Threshold Values
GS80 with Only One Threshold Value

Considering a stringent RI threshold of 80% as previously described in Carayol et al (2011) (GS80%), 57 SNPs (see Table 1) were distributed in two gender-specific clusters of 31 SNPs (5 SNPs were present in the two clusters, see Tables 5 and 6) with an average RI of 92% and 93% in the discovery and validation samples, respectively. AUCs for this genetic model were similar in both the discovery (0.70, 95% CI: 0.65-0.75, for males and 0.76, 95% CI: 0.71-0.81, for females) and the validation populations (0.70, 95% CI: 0.65-0.74, for males and 0.73, 95% CI: 0.67-0.79, for females) indicating that this model was stable, i.e. it has the same discriminative ability in two independent populations.

If such an additive default model is assumed for all 31 SNPs in males genetic score and 31 SNPs in female genetic score, AUC are estimated to 0.68 (p=4.9×10⁻¹¹) and 0.72 (p=5.1×10-12) in males and females respectively in the discovery population and 0.65 (p=1.5×10-7) in males and 0.64 (p=3.09×10-5) in females in the validation population, showing that lower but significant performance is obtained using a simple additive default model.

Genetic scores of the affected individuals and their unaffected siblings in the validation population ranged from 22 to 48 in males, and from 22 to 45 in females. To evaluate the discriminative performance of the genetic score model GS80%, specificity, sensitivity, and positive predictive values (PPV) were estimated for different genetic score thresholds (see Tables 7 and 8).

In males, assuming a 25.9% prevalence in males siblings of children with autism, any threshold value above 27 (GS≧27) is associated to a significant increase in risk or positive predictive value (95% confidence intervals did not include 25.9% in Table 7) and could be used as threshold value. If one want to limit the number of false positive to 20% (i.e. high specificity), a threshold value of 37 or higher allows to identify children with a risk of autism (i.e. positive predictive value) of 43.7% (GS=37 threshold value) or higher (any GS threshold value above 37).

At a threshold of 37 points, the model identified half of the affected individuals (sensitivity=48%, 95% CI: 44%-51%) while limiting the number of false positives to 21% (specificity=79%, 95% CI: 70%-86%). Using a higher genetic score threshold of 40 points dramatically decreased the number of false positives to 7% (specificity=93%, 95% CI: 88%-98%) and identified 20% of affected children (sensitivity=20%, 95% CI: 17%-23%). This genetic score threshold was associated with a PPV of 51% (95% CI: 38%-73%) which was twice as high as the reported 25.9% male sibling recurrence risk (Ozonoff et al, 2011). Further values of sensitivity, specificity and PPV for other single threshold values are provided in following Table 7 for the GS80 in males in the validation population.

TABLE 7
Discriminative performance of the genetic score model
GS80% in males from the validation population

| Genetic | | | | | | |
|---|---|---|---|---|---|---|
| score | Sensi- | | Speci- | | Positive | |
| thresh- | tivity | | ficity | | Predictive | |
| olds | (95% CI) | | (95% CI) | | Value (95% CI) | |
| 22 | 100% | (—) | 0% | (—) | 25.9% | (—) |
| 23 | 100% | (99-100) | 0% | (—) | 25.9% | (—) |
| 24 | 100% | (99-100) | 1% | (0-3) | 26.0% | (25.8-26.4) |
| 25 | 100% | (99-100) | 2% | (0-4) | 26.1% | (25.8-26.7) |
| 26 | 99% | (98-100) | 4% | (0-8) | 26.5% | (25.8-27) |
| 27 | 99% | (98-100) | 5% | (2-9) | 26.6% | (26.0-27.3) |
| 28 | 98% | (97-99) | 8% | (4-14) | 27.2% | (26.2-28.3) |
| 29 | 97% | (95-98) | 14% | (8-20) | 28.2% | (26.9-29.8) |
| 30 | 95% | (93-97) | 15% | (9-22) | 28.1% | (26.7-29.7) |
| 31 | 91% | (89-93) | 22% | (15-30) | 29.1% | (27.3-31.3) |
| 32 | 87% | (85-90) | 33% | (25-42) | 31.3% | (28.9-34.8) |
| 33 | 81% | (78-84) | 45% | (35-54) | 33.9% | (30.6-38.1) |
| 34 | 74% | (71-78) | 55% | (45-64) | 36.4% | (32.2-41.7) |
| 35 | 67% | (64-71) | 66% | (57-75) | 41.0% | (35.2-47.8) |
| 36 | 58% | (54-61) | 73% | (64-81) | 42.4% | (35.9-50.7) |
| 37 | 48% | (44-51) | 79% | (70-86) | 43.7% | (36.8-53.7) |
| 38 | 39% | (35-43) | 84% | (77-90) | 45.2% | (36.8-57.7) |
| 39 | 27% | (24-31) | 90% | (84-95) | 49.1% | (38.0-65.6) |
| 40 | 20% | (17-23) | 93% | (88-98) | 51.3% | (37.8-73.1) |
| 41 | 14% | (11-17) | 94% | (90-98) | 46.2% | (32.9-72.0) |
| 42 | 10% | (7-12) | 97% | (93-100) | 50.0% | (33.3-100.0.0) |
| 43 | 6% | (4-8) | 100% | (—) | 100% | (—) |
| 44 | 3% | (2-4) | 100% | (—) | 100% | (—) |
| 45 | 1% | (1-2) | 100% | (—) | 100% | (—) |
| 46 | 1% | (0-1) | 100% | (—) | 100% | (—) |
| 47 | 1% | (0-1) | 100% | (—) | 100% | (—) |
| 48 | 0% | (—) | 100% | (—) | 100% | (—) |

Following the same reasoning as for male, assuming a 9.6% prevalence in female siblings of children with autism, any threshold value above 24 (GS≧24) is associated to a significant increase in risk or positive predictive value (95% confidence intervals did not include 9.6% in Table 8) and could be used a threshold value. If one want to limit the number of false positive to 20% (i.e. high specificity), a threshold value of 36 or higher allow to identify children with a risk of autism (i.e. positive predictive value) of 23.7% (GS=36 threshold value) or higher (any GS threshold value above 36).

More than half of affected female individuals (sensitivity=52%, 95% CI: 45%-60%) had a genetic score higher than 36 points whereas less than 20% of unaffected individuals exceeded this threshold (specificity=82%, 95% CI: 76%-88%). A PPV of 27% (95% CI: 19%-42%), which represented almost three times the reported female sibling recurrence risk of 9.6% (Ozonoff et al, 2011), was reached at a genetic score threshold of 37 points and was associated with a sensitivity of 39% (95% CI: 32%-47%) and a specificity of 89% (95% CI: 83%-94%). Further values of sensitivity, specificity and PPV for other single threshold values are provided in following Table 8 for the GS80 in females in the validation population.

TABLE 8
Discriminative performance of the genetic score model
GS80% in females from the validation population

| Genetic | | | | | | |
|---|---|---|---|---|---|---|
| score | Sensi- | | Speci- | | Positive | |
| thresh- | tivity | | ficity | | Predictive | |
| olds | (95% CI) | | (95% CI) | | Value (95% CI) | |
| 22 | 100% | (—) | 0% | (—) | 9.6% | (—) |
| 23 | 100% | (—) | 1% | (0-2) | 9.7% | (9.6-9.8) |
| 24 | 100% | (—) | 2% | (0-4) | 9.7% | (9.7-9.9) |
| 25 | 99% | (98-100) | 2% | (0-4) | 9.7% | (9.8-9.8) |
| 26 | 99% | (98-100) | 3% | (1-6 ) | 9.8% | (9.6-10.1) |
| 27 | 99% | (97-100) | 5% | (2-9) | 10.0% | (10.7-10.3) |
| 28 | 99% | (97-100) | 7% | (3-12) | 10.2% | (9.8-10.6) |
| 29 | 97% | (94-99) | 17% | (11-23) | 11.1% | (10.3-11.9) |
| 30 | 94% | (90-98) | 27% | (19-34) | 12.0% | (10.9-13.2) |
| 31 | 90% | (85-95) | 36% | (27-44) | 12.9% | (11.5-14.7) |
| 32 | 82% | (76-88) | 42% | (33-51) | 13.1% | (11.3-15.3) |
| 33 | 75% | (68-81) | 54% | (45-63) | 14.8% | (12.5-17.8) |
| 34 | 67% | (60-74) | 65% | (57-73) | 16.9% | (14.0-21.2) |
| 35 | 58% | (50-66) | 70% | (62-78) | 17.2% | (13.7-22.2) |
| 36 | 52% | (45-60) | 82% | (76-88) | 23.8% | (17.6-33.0) |
| 37 | 39% | (32-47) | 89% | (83-94) | 27.1% | (18.6-41.6) |
| 38 | 28% | (21-35) | 96% | (92-99) | 40.0% | (24.8-67.3) |
| 39 | 20% | (14-27) | 99% | (65-100) | 74.5% | (45.7-100.0) |
| 40 | 15% | (10-21) | 100% | (—) | 100% | (—) |
| 41 | 10% | (5-15) | 100% | (—) | 100% | (—) |
| 42 | 7% | (3-11) | 100% | (—) | 100% | (—) |
| 43 | 5% | (2-8) | 100% | (—) | 100% | (—) |
| 44 | 2% | (1-5) | 100% | (—) | 100% | (—) |
| 45 | 1% | (0-2) | 100% | (—) | 100% | (—) |

To ascertain that the genetic score models were not associated with autism by chance, male genetic score models were applied to females and vice versa. In this configuration, no association was observed and AUCs were not significantly different from noninformativity (AUC=0.5). Specifically, assuming the same stringent RI threshold of 80%, AUCs were estimated to be 0.47 (P=0.30) and 0.47 (P=0.34) in males and females, respectively.

GS80 with Several Threshold Values

A multi-risk class test may be constructed using more than one threshold value. Two threshold values may be set to create 3 classes of risk: a reference class where the risk is close or equal to the prevalence of the disease, a low risk class where the risk is lower than the risk in the reference class, and a high risk class where the risk is higher than in the reference class.

Example in females with two threshold values:

Using two threshold values in females (GS=32 and 37), three classes are delineated: a first class defined as the reference class (GS<37 and GS≧32) where the risk is similar to the prevalence in siblings 9.6%; a second class of lower risk (GS<32) where the probability to be affected when the GS is lower than 32 is 3%; and a third risk class of higher risk class (GS≧37), where the probability to be affected when the GS is higher than 37 is 27%.

Then 3, 4 or more threshold values can also be applied.

Example in males with 4 threshold values:

Five classes are delineated using 4 GS threshold values (30, 35, 40 and 45): a reference class (GS≧35 and GS<40) where the risk is close to the prevalence of the disease; a high risk class (GS≧40 and GS<45), where the risk is 49% and a very high risk class where the risk is 100%; a low risk class (GS≧30 and GS<35) were the risk is 16% and a very low risk class (GS<30) were the risk is 8%.

The number and the value of the different threshold values are settled according to the performance and characteristics expected for the test defined by risk in classes, sensitivity and specificity.

### Further Genetic Scores (GS85, GS90, GS95)

Subgroups were then defined according to RI values for the best fitted genetic model in the Discovery and Validation sample. SNPs with a RI for a given genetic model greater than a fixed value in both samples were selected to build the genetic score. The process was applied in males and in females separately to construct two different genetic score, one in males and one in females. Three different RI value defining three different degrees of reproducibility of the SNPs have been chosen: 0.95, 0.90 and 0.85. AUCs and associated p-value have been provided for the different genetic scores in males (Table 5) and females (Table 6).

GS80 Outperforms the Test Based on Genotyping of 4 or 8 SNPs Previously Described in Carayol et al, 2010 and Carayol et al, 2011, Respectively, and May be Combined with this Test for Improved Reliability

4 and 8 SNPs genetic score models are proposed in Carayol et al. (2010) and Carayol et al. (2011) respectively. The area under the curve (AUC) is equal to the probability that a genetic score will rank a randomly chosen affected patient higher than a randomly chosen unaffected individual. AUCs were estimated to 0.59 (no gender difference) for the 4 SNPs genetic score (Carayol et al. 2010) and, 0.59 and 0.66 in males and females respectively for a 8 SNPs gender specific genetic model (Carayol et al. 2011). Using 57 SNPs, AUCs increased to 0.7 in males and 0.73 in females in the validation population. Despite the unambiguous interest and good performances of the previously described tests based on genotyping of 4 and 8 SNPs, the new test according to the invention, based on analysis of 57 SNPs, is even more reliable.

In addition, the new test according to the invention may be combined with the previously described test based on genotyping of 8 SNPs, resulting in further slightly improved reliability.

Tables 9 and 10 provide sensitivity, specificity as positive and negative predictive value for a 65 SNPs gender specific genetic score model in males and females. In males, the prevalence of autism in siblings of affected children is estimated to 25.9%. Using a genetic score threshold of 46 allow to identified 40% of siblings (sensitivity) with a two-fold increase in risk (47.9% positive predictive value) with only 15% of false positive results (1 minus the specificity). The prevalence in female is estimated to 9.6%. Use of a 42 genetic score threshold identify 65% of siblings (sensitivity) with a two-fold increased risk (22.5%) and less than 25% (1 minus specificity) false positive results. With a 48 genetic score threshold, 14% of siblings (sensitivity) with more than 50% risk (positive predictive value) are assessed and only 3% of false positive results expected (1 minus specificity).

TABLE 9
Sensitivity, specificity as positive and negative predictive
value for a 37 SNPs genetic score model in males

| Genetic | | | | |
|---|---|---|---|---|
| Score | | | Positive | Negative |
| Thresh- | Sensi- | Speci- | Predictive | Predictive |
| old | tivity | ficity | Value | Value |
| 30 | 100.0% | 0.0% | 25.9% | 100.0% |
| 31 | 100.0% | 0.8% | 26.0% | 100.0% |
| 32 | 100.0% | 1.5% | 26.2% | 100.0% |
| 33 | 99.9% | 2.3% | 26.3% | 97.9% |
| 34 | 99.2% | 3.0% | 26.3% | 91.0% |
| 35 | 98.7% | 5.3% | 26.7% | 92.2% |
| 36 | 97.7% | 9.8% | 27.5% | 92.5% |
| 37 | 96.6% | 15.8% | 28.6% | 93.0% |
| 38 | 93.8% | 21.8% | 29.5% | 91.0% |
| 39 | 90.8% | 30.8% | 31.5% | 90.6% |
| 40 | 85.3% | 36.8% | 32.1% | 87.8% |
| 41 | 79.4% | 45.9% | 33.9% | 86.4% |
| 42 | 72.8% | 54.9% | 36.1% | 85.2% |
| 43 | 64.6% | 63.9% | 38.5% | 83.8% |
| 44 | 57.7% | 71.4% | 41.4% | 82.8% |
| 45 | 49.1% | 78.9% | 44.9% | 81.6% |
| 46 | 39.6% | 85.0% | 47.9% | 80.1% |
| 47 | 32.3% | 90.2% | 53.6% | 79.2% |
| ≧48 | 25.8% | 96.2% | 70.6% | 78.8% |

TABLE 10
Sensitivity, specificity as positive and negative predictive
value for a 36 SNPs genetic score model in females

| Genetic | | | | |
|---|---|---|---|---|
| Score | | | Positive | Negative |
| Thresh- | Sensi- | Speci- | Predictive | Predictive |
| old | tivity | ficity | Value | Value |
| 29 | 100.0% | 0.0% | 9.6% | 100.0% |
| 30 | 100.0% | 1.3% | 9.7% | 100.0% |
| 31 | 100.0% | 2.5% | 9.8% | 100.0% |
| 32 | 100.0% | 3.1% | 9.9% | 100.0% |
| 33 | 100.0% | 4.4% | 10.0% | 100.0% |
| 34 | 99.4% | 6.3% | 10.1% | 99.1% |
| 35 | 97.8% | 11.3% | 10.5% | 98.0% |
| 36 | 95.6% | 20.1% | 11.3% | 97.7% |
| 37 | 93.4% | 30.8% | 12.5% | 97.8% |
| 38 | 91.2% | 36.5% | 13.2% | 97.5% |
| 39 | 86.7% | 44.7% | 14.3% | 96.9% |
| 40 | 81.2% | 56.6% | 16.6% | 96.6% |
| 41 | 73.5% | 67.9% | 19.6% | 96.0% |
| 42 | 65.2% | 76.1% | 22.5% | 95.4% |
| 43 | 54.1% | 82.4% | 24.6% | 94.4% |
| 44 | 44.8% | 84.9% | 23.9% | 93.5% |
| 45 | 33.7% | 89.3% | 25.1% | 92.7% |
| 46 | 24.9% | 93.1% | 27.6% | 92.1% |
| 47 | 16.0% | 97.5% | 40.3% | 91.6% |
| 48 | 13.8% | 98.7% | 53.8% | 91.5% |
| 49 | 10.5% | 99.4% | 63.9% | 91.3% |
| ≧50 | 7.2% | 100.0% | 100.0% | 91.0% |

A Particular SNP of Interest May be Replaced by Another SNP in Linkage Disequilibrium with this SNP of Interest

SNP rs7172184 belongs to the genetic score in males and females. If this SNP is replaced by rs2018052, a SNP in linkage disequilibrium (r2=0.815 as defined in HapMap), based on the discovery population, the AUCs are estimated to 0.70 (p=6.06×10⁻¹⁴) in males and 0.75 (p=5.7×10⁻¹⁶) in females instead of 0.70 and 0.76 with the original SNP (rs7172184).

### REFERENCES

Abrahams B S, Geschwind D H. Advances in autism genetics: on the threshold of a new neurobiology. Nat Rev Genet. 2008 May; 9(5):341-55.
Anney R, et al. A genome-wide scan for common alleles affecting risk for autism. Hum Mol Genet. 2010 Oct. 15; 19(20):4072-82.
Barton, M. L., T. Dumont-Mathieu, et al. (2011). “Screening Young Children for Autism Spectrum Disorders in Primary Practice.” J Autism Dev Disord.
Campbell D B, Li C, Sutcliffe J S, Persico A M, Levitt P. Genetic evidence implicating multiple genes in the MET receptor tyrosine kinase pathway in autism spectrum disorder. Autism Res. 2008 June; 1(3):159-68.
Carayol J, Schellenberg G D, Dombroski B, Genin E, Rousseau F, Dawson G. Autism risk assessment in siblings of affected children using sex-specific genetic scores. Mol Autism 2011; 2(1): 17.
Carayol J, Schellenberg G D, Tores F, Hager J, Ziegler A, Dawson G. Assessing the impact of a combined analysis of four common low-risk genetic variants on autism risk. Mol Autism 2010; 1(1): 4.
CDC, C. f. D. C. a. P. (2012). “Prevalence of Autism Spectrum Disorders—Autism and Developmental Disabilities Monitoring Network, 14 Sites, United States, 2008.” Morbidity and Mortality Weekly Report Surveillance Summary 61(3).
Chamak, B., B. Bonniau, et al. (2011). “The autism diagnostic experiences of French parents.” Autism 15(1): 83-97.
Coutinho A M, Sousa I, Martins M, Correia C, Morgadinho T, Bento C, Marques C, Ataide A, Miguel T S, Moore J H, Oliveira G, Vicente A M. Evidence for epistasis between SLC6A4 and ITGB3 in autism etiology and in the determination of platelet serotonin levels. Hum Genet. 2007 April; 121(2):243-56.
Dawson, G., E. J. Jones, et al. (2012). “Early behavioral intervention is associated with normalized brain activity in young children with autism.” J Am Acad Child Adolesc Psychiatry 51(11): 1150-1159.
Dawson, G. and J. Osterling (1997). Early intervention in autism: Effectiveness and common elements of current approaches. The effectivness of early intervention: second generation research. M. J. Guralnick. Baltimore, Paul H. Brookes: 307-326.
Dawson, G., E. J. Jones, et al. (2012). “Early behavioral intervention is associated with normalized brain activity in young children with autism.” J Am Acad Child Adolesc Psychiatry 51(11): 1150-1159.
Eikeseth S. Outcome of comprehensive psycho-educational interventions for young children with autism. Research in Developmental Disabilities 2009; 30:158-78.
Ganz, M. L. (2007). “The lifetime distribution of the incremental societal costs of autism.” Arch Pediatr Adolesc Med 161(4): 343-349.
Gharani N, Benayed R, Mancuso V, Brzustowicz L M, Millonig J H. Association of the homeobox transcription factor, ENGRAILED 2, 3, with autism spectrum disorder. Mol Psychiatry. 2004 May; 9(5):474-84.
Gotham, K., S. Risi, et al. (2007). “The Autism Diagnostic Observation Schedule: revised algorithms for improved diagnostic validity.” J Autism Dev Disord 37(4): 613-627.
Hallmayer J, Cleveland S, Torres A, Phillips J, Cohen B, Torigoe T et al. Genetic heritability and shared environmental factors among twin pairs with autism. Arch Gen Psychiatry 2011; 68(11): 1095-1102.
Hussman J P, Chung R H, Griswold A J, Jaworski J M, Salyakina D, Ma D, Konidari I, Whitehead P L, Vance J M, Martin E R, Cuccaro M L, Gilbert J R, Haines J L, Pericak-Vance M A. A noise-reduction GWAS analysis implicates altered regulation of neurite outgrowth and guidance in autism. Mol Autism. 2011 Jan. 19; 2(1):1.
Jacobson, J. W. and J. A. Mulick (2000). “System and cost research issues in treatments for people with autistic disorders.” J Autism Dev Disord 30(6): 585-593.
Johnson, C. P. and S. M. Myers (2007). “Identification and evaluation of children with autism spectrum disorders.” Pediatrics 120(5): 1183-1215.
Kim, S. H., A. Thurm, et al. (2012). “Multisite Study of New Autism Diagnostic Interview-Revised (ADI-R) Algorithms for Toddlers and Young Preschoolers.” J Autism Dev Disord.
Laird N M, Horvath S, Xu X. Implementing a unified approach to family-based tests of association. Genet Epidemiol 2000; 19 Suppl 1: S36-42.
Lajonchere C M. Changing the landscape of autism research: the autism genetic resource exchange. Neuron 2010; 68(2): 187-191.
Lord C, Rutter M, Le Couteur A (1994) Autism Diagnostic Interview-Revised: a revised version of a diagnostic interview for caregivers of individuals with possible pervasive developmental disorders. J Autism Dev Disord 24:659-685
Lovaas O I. Teaching Developmentally Disabled Children: The Me Book. Baltimore, Md.: University Park, 1981.
Lovaas O I. Behavioral treatment and normal educational and intellectual functioning in young autistic children. Journal of Consulting and Clinical Psychology 1987; 55(1): 3-9.
Luyster, R., K. Gotham, et al. (2009). “The Autism Diagnostic Observation Schedule-toddler module: a new module of a standardized diagnostic measure for autism spectrum disorders.” J Autism Dev Disord 39(9): 1305-1320.
Ma S. Empirical study of supervised gene screening. BMC Bioinformatics 2006; 7: 537.
Maniatis, N., et al., The first linkage disequilibrium (LD) maps: delineation of hot and cold blocks by diplotype analysis. Proc Natl Acad Sci USA 99:2228-2233 (2002)
Marui T, Funatogawa I, Koishi S, Yamamoto K, Matsumoto H, Hashimoto O, Nanba E, Nishida H, Sugiyama T, Kasai K, Watanabe K, Kano Y, Sasaki T, Kato N. Association of the neuronal cell adhesion molecule (NRCAM) gene variants with autism. Int J Neuropsychopharmacol. 2009 February; 12(1):1-10.
Martin C L, Duvall J A, Ilkin Y, Simon J S, Arreaza M G, Wilkes K, Alvarez-Retuerto A, Whichello A, Powell C M, Rao K, Cook E, Geschwind D H. Cytogenetic and molecular characterization of A2BP1/FOX1 as a candidate gene for autism. Am J Med Genet B Neuropsychiatr Genet. 2007 Oct. 5; 144B(7):869-76.
Maurice C, Green G, Luce S C. Behavioral Intervention for Young Children with Autism: A Manual for Parents and Professionals. Austin, Tex.: Pro-Ed, 1996.
McCarthy M I, Abecasis G R, Cardon L R, Goldstein D B, Little J, loannidis J P et al. Genome-wide association studies for complex traits: consensus, uncertainty and challenges. Nat Rev Genet 2008; 9(5): 356-369.
Melke J, Goubran Botros H, Chaste P, Betancur C, Nygren G, Anckarsäter H, Rastam M, Stählberg O, Gillberg I C, Delorme R, Chabane N, Mouren-Simeoni M C, Fauchereau F, Durand C M, Chevalier F, Drouot X, Collet C, Launay J M, Leboyer M, Gillberg C, Bourgeron T. Abnormal melatonin synthesis in autism spectrum disorders. Mol Psychiatry. 2008 January; 13(1):90-8.
Morrow E M, Yoo S Y, Flavell S W, Kim T K, Lin Y, Hill R S, Mukaddes N M, Balkhy S, Gascon G, Hashmi A, Al-Saad S, Ware J, Joseph R M, Greenblatt R, Gleason D, Ertelt J A, Apse K A, Bodell A, Partlow J N, Barry B, Yao H, Markianos K, Ferland R J, Greenberg M E, Walsh C A. Identifying autism loci and genes by tracing recent shared ancestry. Science. 2008 Jul. 11; 321(5886):218-23.
Myakishev M V, Khripin Y, Hu S, Hamer D H, High-throughput SNP genotyping by allele-specific PCR with universal energy-transfer-labeled primers, Genome Res, no 11, pp 163-169, (2001)
Nazarenko I A, Bhatnagar S K, Hohman R J. A closed tube format for amplification and detection of DNA based on energy transfer. Nucleic Acids Res, no 25, pp. 2516-2521. (1997)
Ozonoff S, Young G S, Carter A, Messinger D, Yirmiya N, Zwaigenbaum L et al. Recurrence Risk for Autism Spectrum Disorders: A Baby Siblings Research Consortium Study. Pediatrics 2011.
Patel S D, Le-Niculescu H, Koller D L, Green S D, Lahiri D K, McMahon F J et al. Coming to grips with complex disorders: genetic risk prediction in bipolar disorder using panels of genes identified through convergent functional genomics. Am J Med Genet B Neuropsychiatr Genet 2010; 153B(4): 850-877
Pereira, T. V., N. A. Patsopoulos, et al. (2009). “Discovery properties of genome-wide association signals from cumulatively combined data sets.” Am J Epidemiol 170(10): 1197-1206.
Philippi A, Tores F, Carayol J, Rousseau F, Letexier M, Roschmann E, Lindenbaum P, Benajjou A, Fontaine K, Vazart C, Gesnouin P, Brooks P, Hager J. Association of autism with polymorphisms in the paired-like homeodomain transcription factor 1 (PITX1) on chromosome 5q31: a candidate gene analysis. BMC Med Genet. 2007 Dec. 6; 8:74.
Reich, D E et al, Linkage disequilibrium in the human genome. Nature 411:199-204 (2001)
Reichow, B., E. E. Barton, et al. (2012). “Early intensive behavioral intervention (EIBI) for young children with autism spectrum disorders (ASD).” Cochrane Database Syst Rev 10: CD009260.
Rice C E: Prevalence of autism spectrum disorders—Autism and Developmental Disabilities Monitoring Network, United States, 2006. MMWR Surveill Summ 2009, 58:1-20.
Risch, N. & Merkiangas, K, The future of genetic studies of complex human diseases. Science 273:1516-1517 (1996)
Robins, D. L., D. Fein, et al. (2001). “The Modified Checklist for Autism in Toddlers: an initial study investigating the early detection of autism and pervasive developmental disorders.” J Autism Dev Disord 31(2): 131-144.
Scherer S W, Dawson G. Risk factors for autism: translating genomic discoveries into diagnostics. Hum Genet. 2011 July; 130(1):123-48.
Shao Y, Raiford K L, Wolpert C M, Cope H A, Ravan S A, Ashley-Koch A A et al. Phenotypic homogeneity provides increased support for linkage on chromosome 2 in autistic disorder. Am J Hum Genet 2002; 70(4): 1058-1061.
Shattuck, P. T., M. Durkin, et al. (2009). “Timing of identification among children with an autism spectrum disorder: findings from a population-based surveillance study.” J Am Acad Child Adolesc Psychiatry 48(5): 474-483.
Smith M, Rogers S, Dawson G. The Early Start Denver Model: a comprehensive early intervention approach for toddlers with autism. In: Handleman J S, Harris S L, eds. Preschool Education Programs for Children With Autism. 3rd ed. Austin, Tex.: Pro-Ed Corporation, Inc; 2008:65-101
Smith T. Early and intensive behavioral intervention in autism. In: Weisz J R, Kazdin A E editor(s). Evidence-Based Psychotherapies for Children and Adolescents. 2nd Edition. New York: Guilford, 2010:312-26.
Stone J L, Merriman B, Cantor R M, Yonan A L, Gilliam T C, Geschwind D H et al. Evidence for sex-specific risk alleles in autism spectrum disorder. Am J Hum Genet 2004; 75(6): 1117-1123.
US2011/0086777
US2011/0207124
Veenstra-VanderWeele J, Cook E H, Jr. (2004) Molecular genetics of autism spectrum disorder. Mol Psychiatry 9:819-832
Voineagu I, Wang X, Johnston P, Lowe J K, Tian Y, Horvath S, Mill J, Cantor R M, Blencowe B J, Geschwind D H. Transcriptomic analysis of autistic brain reveals convergent molecular pathology. Nature. 2011 May 25; 474(7351):380-4.
Voineagu I. Gene expression studies in autism: moving from the genome to the transcriptome and beyond. Neurobiol Dis. 2012 January; 45(1):69-75.
Wang K, Zhang H, Ma D, Bucan M, Glessner J T, Abrahams B S et al. Common genetic variants on 5p14.1 associate with autism spectrum disorders. Nature 2009; 459(7246): 528-533. (a)
Wang, J., Lin, M., Crenshaw, A., Hutchinson, A., Hicks, B., Yeager, M., Berndt, S., Huang, W., Hayes, R. B., Chanock, S. J., Jones, R. C., and Ramakrishnan, R. High-throughput single nucleotide polymorphism genotyping using nanofluidic Dynamic Arrays. BMC Genomics. N) 10 p 561 (2009) (b)
Warren, Z., M. L. McPheeters, et al. (2011). “A systematic review of early intensive intervention for autism spectrum disorders.” Pediatrics 127(5): e1303-1311.
Weiss L A, Arking D E; Gene Discovery Project of Johns Hopkins & the Autism Consortium, Daly M J, Chakravarti A. A genome-wide linkage and association scan reveals novel loci for autism. Nature. 2009 Oct. 8; 461(7265):802-8.
WO2009/043178,
WO2011/031786
WO2011/138372
Zeger S L, Liang K Y. Longitudinal data analysis for discrete and continuous outcomes. Biometrics 1986; 42(1): 121-130.
Zwaigenbaum, L. (2010). “Advances in the early detection of autism.” Curr Opin Neurol 23(2): 97-102

1. A method of determining a risk of developing autism in a subject, the method comprising obtaining a biological sample from a subject and detecting in the biological sample a number of autism-associated risk alleles by genotyping a single nucleotide polymorphism (SNP) in the gene loci of at least HTR5A, MACF1, RBFOX1, ABR, PTPRG, CACNA2D1, GFRA1, DSCAML1, CHRM3, LPPR4, DLG2, SLC9A9, and BASP1 in the biological sample,
wherein the SNP in HTR5A is rs893109, the SNP in MACF1 is rs260969, the SNP in RBFOX1 is rs12925135, the SNP in ABR is rs2663327, the SNP in PTPRG is rs636624, the SNP in CACNA2D1 is rs2367910, the SNP in GFRA1 is rs10787637, the SNP in DSCAML1 is rs695083, the SNP in CHRM3 is rs10802802, the SNP in LPPR4 is rs712886, the SNP in DLG2 is rs12275631, the SNP in SLC9A9 is rs3928471 and the SNP in BASP1 is rs298542, or a SNP in each of the gene loci in linkage disequilibrium with each of the aforementioned SNPs with an r²≧0.80; the genotyping is carried out by sequencing, selective hybridization, or selective amplification; and the risk of developing autism is determined based on the number of autism-associated risk alleles detected in the biological sample.

2. The method of claim 1, further comprising detecting autism-associated risk alleles by genotyping:
a SNP in the gene loci of any or all of KCNIP1, UGCG, NTRK3, PLCB1, NELL1, GPR98, MAGI2, PLAGL1, CNTN6, DLG4, ERC2, TRIM9, SYT14, JARID2, CDH13, SULF2, GRIN2A and NRG3, or combinations thereof; or
a SNP in the gene loci of any or all of NRG1, TRIM2, EPHA5, PCDH10, HIP1, APBA1, PDE4D and EGLN3, or combinations thereof.

3. The method of claim 1, further comprising detecting autism-associated risk alleles by genotyping at least one SNP in the gene loci selected from the group consisting of ACCN1, AKAP7, APBA1, ASTN2, CADM1, CDH13, CNTN6, DCLK1, DCLK2, DLG4, EGLN3, EPHA5, ERC2, GPR98, GRIN2A, GRIN2B, GRM7, HIP1, JARID2, KCNH5, KCNIP1, MAGl2, MAP1S, MAP2K1, NAV2, NELL1, NRG1, NRG3, NTRK3, PAX2, PCDH10, PDE11A, PDE4D, PLAGL1, PLCB1, PTPRD, RGS6, SLC24A2, SULF2, SYT14, TRIM2, TRIM9 and UGCG, or combinations thereof.

4. (canceled)

5. The method of claim 2, wherein the SNP in KCNIP1 is rs12514116, the SNP in UGCG is rs16916456, the SNP in NTRK3 is rs7172184, the SNP in PLCB1 is rs8123323, the SNP in NELL1 is rs10766739, the SNP in GPR98 is rs16868972, the SNP in MAGl2 is rs12535987, the SNP in PLAGL1 is rs2076683, the SNP in CNTN6 is rs9837484, the SNP in DLG4 is rs314253, the SNP in ERC2 is rs1485677, the SNP in TRIM9 is rs10150121, the SNP in SYT14 is rs7534723, the SNP in JARID2 is rs9370809, the SNP in CDH13 is rs9940922, the SNP in SULF2 is rs6063144, the SNP in GRIN2A is rs4782109, the SNP in NRG3 is rs2820100 or rs7075400, the SNP in NRG1 rs723811, the SNP in TRIM2 is rs11942354, the SNP in EPHA5 is rs1597611, the SNP in PCDH10 is rs4404561, the SNP in HIP1 is rs6962352, the SNP in APBA1 is rs11139294, the SNP in PDE4D is rs35284, and the SNP in EGLN3 is rs946630.

6. (canceled)

7. The method of claim 3, wherein the SNP in KCNH5 is rs1041644, the SNP in MAP1S is rs12985015, the SNP in GRM7 is rs1569284, the SNP in PAX2 is rs2077642, the SNP in PTPRD is rs2382104, the SNP in PDE11A is rs2695112, the SNP in RGS6 is rs6574041, the SNP in ASTN2 is rs7021928, the SNP in ACCN1 is rs7225320, the SNP in DCLK2 is rs9307866, the SNP in SLC24A2 is rs957910, the SNP in AKAP7 is rs6923644, the SNP in DCLK1 is rs1556060, the SNP in MAP2K1 is rs1432443, the SNP in CADM1 is rs220836, the SNP in GRIN2B is rs7974275, and the SNP in NAV2 is rs10500866.

8. (canceled)

9. The method of claim 1, further comprising detecting autism-associated risk alleles by genotyping a SNP in the gene loci of any or all of PITX1, ATP2B2, EN2, JARID2, MARK1, ITGB3, CNTNAP2, and HOXA 1, or combinations thereof.

10. A method of determining a risk of developing autism in a male subject, the method comprising obtaining a biological sample from a male subject and detecting in the biological sample a number of autism-associated risk alleles by genotyping SNPs in the gene loci of at least HTR5A, MACF1, RBFOX1, ABR, PTPRG, and CACNA2D1, in the biological sample, wherein said SNPs are rs893109, rs260969, rs12925135, rs2663327, rs636624 and rs2367910 or SNPs in linkage disequilibrium with each of the aforementioned SNPs with an r²≧0.80; the genotyping is carried out by sequencing, selective hybridization, or selective amplification; and the risk of developing autism is determined based on the number of autism-associated risk alleles detected in the biological sample.

11. The method of claim 10, further comprising detecting autism-associated risk alleles by genotyping a SNP in the gene loci of any or all of KCNIP1, UGCG, NTRK3, PLCB1, NELL1, GPR98, MAGl2, and PLAGL1, or combinations thereof.

12. The method of claim 10, further comprising detecting autism-associated risk alleles by genotyping a SNP in the gene loci of any or all of NRG1, TRIM2, EPHA5, PCDH10, and HIP1, or combinations thereof.

13. The method of claim 10, further comprising detecting autism-associated risk alleles by genotyping a SNP in the gene loci of any or all of PDE11A, AKAP7, DCLK1, KCNH5, GRIN2A, ACCN1, DCLK2, ASTN2, GRM7, MAP2K1, CADM1, and GRIN2B, or combinations thereof.

14. The method of claim 10, further comprising detecting autism-associated risk alleles by genotyping a SNP in the gene loci of any or all of PITX1, ATP2B2, EN2, JARID2, CNTNAP2, and HOXA 1, or combinations thereof.

15. A method of determining a risk of developing autism in a female subject, the method comprising obtaining a biological sample from a female subject and detecting in the biological sample a number of autism-associated risk alleles by genotyping SNPs in the gene loci of at least CHRM3, DSCAML1, PTPRG, GFRA1, LPPR4, DLG2, SLC9A9 and BASP1, in the biological sample, wherein said SNPs are rs10802802, rs695083, rs636624, rs10787637, rs712886, rs12275631, rs3928471 and rs298542 or SNPs in linkage disequilibrium with each of the aforementioned SNPs with an r²≧0.80; the genotyping is carried out by sequencing, selective hybridization, or selective amplification; and the risk of developing autism is determined based on the number of autism-associated risk alleles detected in the biological sample.

16. The method of claim 15, further comprising detecting autism-associated risk alleles by genotyping a SNP in the gene loci of any or all of CNTN6, NTRK3, DLG4, ERC2, TRIM9, SYT14, JARID2, CDH13, SULF2, GRIN2A and NRG3, or combinations thereof.

17. The method of claim 15, further comprising detecting autism-associated risk alleles by genotyping a SNP in the gene loci of any or all of APBA1, ABR, NRG3, PDE4D and EGLN3, or combinations thereof.

18. The method of claim 15, further comprising detecting autism-associated risk alleles by genotyping a SNP in the gene loci of any or all of RGS6, SLC24A2, PTPRD, NAV2, PCDH10, MAP1S, and PAX2, or combinations thereof.

19. The method of claim 15, further comprising detecting autism-associated risk alleles by genotyping a SNP in the gene loci of any or all of EN2, JARID2, MARK1, ITGB3, and CNTNAP2, or combinations thereof.

20. (canceled)

21. The method of claim 1, wherein the risk of developing autism is determined depending on the number of autism-associated risk alleles that are detected, by calculating a genetic score.

22. The method of claim 21, wherein the genetic score is compared to one or more threshold values.

23. The method of claim 1, wherein the subject is not related to anyone with an autism-spectrum disorder (ASD) or is a sibling of an individual with an ASD.

24-30. (canceled)

31. A method for treating autism in a subject, the method comprising:
a) determining a risk of developing autism in a subject by the method of claim 1, and
b) if said subject is determined to be at risk of developing autism, then submitting said subject to:
i) a behavioral autism instrument,
ii) an indirect, interview-based autism instrument with third parties,
iii) Early Intensive Behavioural Intervention, or
iv) a combination of at least two of i) to iii).

32. The method of claim 9, wherein the SNP is selected from the group consisting of rs6872664, rs2278556, rs1861972, rs7766973, rs12410279, rs5918, rs7794745, and rs10951154, or combinations thereof.

33. The method of claim 11, wherein the SNP is selected from the group consisting rs12514116, rs16916456, rs7172184, rs8123323, rs10766739, rs16868972, rs12535987 and rs2076683, or combinations thereof

34. The method of claim 12, wherein the SNP is selected from the group consisting of rs723811, rs11942354, rs1597611, rs4404561 and rs6962352, or combinations thereof.

35. The method of claim 13, wherein the SNP is selected from the group consisting of rs2695112, rs6923644, rs1556060, rs1041644, rs4782109, rs7225320, rs9307866, rs7021928, rs1569284, rs1432443, rs220836, and rs7974275, or combinations thereof.

36. The method of claim 14, wherein the SNP is selected from the group consisting of rs6872664, rs2278556, rs1861972, rs7766973, rs7794745, and rs10951154, or combinations thereof.

37. The method of claim 16, wherein the SNP is selected from the group consisting of rs9837484, rs7172184, rs314253, rs1485677, rs10150121, rs7534723, rs9370809, rs9940922, rs6063144, rs4782109 and rs2820100, or combinations thereof.

38. The method of claim 17, wherein the SNP is selected from the group consisting of rs11139294, rs2663327, rs7075400, rs35284 and rs946630, or combinations thereof.

39. The method of claim 18, wherein the SNP is selected from the group consisting of rs6574041, rs957910, rs2382104, rs10500866, rs4404561, rs12985015, and rs2077642, or combinations thereof.

40. The method of claim 19, wherein the SNP is selected from the group consisting of rs1861972, rs7766973, rs12410279, rs5918, and rs7794745, or combinations thereof.


SEQUENCE LISTING (65 sequences)

SEQ ID NO: 1 - DNA, 52 bp, Artificial Sequence
  Context sequence of SNP rs10150121
ctgtgctacttaatgtagaccacctgyttgatttcctgaatgtggtctatgt

SEQ ID NO: 2 - DNA, 52 bp, Artificial Sequence
  Context sequence of SNP rs1041644
caccattttaaagagtttaactaaatmaaattcatcaatgtttccactatgt

SEQ ID NO: 3 - DNA, 52 bp, Artificial Sequence
  Context sequence of SNP rs10766739
acacaattggcaaaaccccctgtcacrgtcagtaaactttgaggacctgctc

SEQ ID NO: 4 - DNA, 52 bp, Artificial Sequence
  Context sequence of SNP rs10787637
tggatagttggactctgcaacctactraaacaaacatgttaaaaattaaaca

SEQ ID NO: 5 - DNA, 52 bp, Artificial Sequence
  Context sequence of SNP rs10802802
tctgtctcatccctggtcaggatgtargtataagtttgaaggatcaaaaaat

SEQ ID NO: 6 - DNA, 52 bp, Artificial Sequence
  Context sequence of SNP rs11139294
tgccctcatggaacttaccctgaggartttacaatagaaataattaaacata

SEQ ID NO: 7 - DNA, 52 bp, Artificial Sequence
  Context sequence of SNP rs12985015
cagcagtcctgagggctcagggttccrttttccccacaaatgccatcatctg

SEQ ID NO: 8 - DNA, 52 bp, Artificial Sequence
  Context sequence of SNP rs1485677
ttttaattaatcctctgcaaggaaccragttttgtttgccatcatctcccca

SEQ ID NO: 9 - DNA, 52 bp, Artificial Sequence
  Context sequence of SNP rs1569284
aaagtctatttattttcccaactaatrtgtgtatgcttcatgagagcacagc

SEQ ID NO: 10 - DNA, 52 bp, Artificial Sequence
  Context sequence of SNP rs1597611
agtgagaagttatggtgctttctctcrcctgcctatggctgcccacagtcca

SEQ ID NO: 11 - DNA, 52 bp, Artificial Sequence
  Context sequence of SNP rs16916456
tgatgtcagtgattgtaactgtcattyctagaacttgtgtggttcttttcat

SEQ ID NO: 12 - DNA, 52 bp, Artificial Sequence
  Context sequence of SNP rs2076683
aatgaactgacactgcacaacaagackgtcacataaaaccacaggaatcact

SEQ ID NO: 13 - DNA, 52 bp, Artificial Sequence
  Context sequence of SNP rs2077642
ctcctgtaggaggcttgagcctgggtytaggttggagacagaggccgagaag

SEQ ID NO: 14 - DNA, 52 bp, Artificial Sequence
  Context sequence of SNP rs2382104
atggacagcctataggctgtaacctgktattaggaataaagctttccctata

SEQ ID NO: 15 - DNA, 52 bp, Artificial Sequence
  Context sequence of SNP rs260969
gtaaaccctcctcctcttttaagtgcygcctcctctgtccttaatgccccca

SEQ ID NO: 16 - DNA, 52 bp, Artificial Sequence
  Context sequence of SNP rs2695112
tcatccccccatgtcgacctaaaagarcacagtttactttttcaaggttctc

SEQ ID NO: 17 - DNA, 52 bp, Artificial Sequence
  Context sequence of SNP rs314253
aagttgagagtttcatgcaaaagaccracccaggggtagtgattctgtggat

SEQ ID NO: 18 - DNA, 52 bp, Artificial Sequence
  Context sequence of SNP rs35284
cgttgtaattctatcttcagaatgatrcattgcaaaagagtgtgacaaaagg

SEQ ID NO: 19 - DNA, 39 bp, Artificial Sequence
  Context sequence of SNP rs3928471
gcttcatgtttatgttctatgttcagyttttggtctgtt

SEQ ID NO: 20 - DNA, 52 bp, Artificial Sequence
  Context sequence of SNP rs4404561
taccagggcctgctcagcaaccagagmagcagaatggggggccagatgcaag

SEQ ID NO: 21 - DNA, 52 bp, Artificial Sequence
  Context sequence of SNP rs4782109
atagcaatgatggacaaactccctgcytcatggggcttgcattttagagcta

SEQ ID NO: 22 - DNA, 52 bp, Artificial Sequence
  Context sequence of SNP rs636624
ttggtcactgcctatttcaattctggrtttctctaacaatgagaaatggtct

SEQ ID NO: 23 - DNA, 52 bp, Artificial Sequence
  Context sequence of SNP rs6574041
gtagtaggatgtttgaagaacagcaargagaccagtgtggctggaagaaggg

SEQ ID NO: 24 - DNA, 52 bp, Artificial Sequence
  Context sequence of SNP rs695083
actggtttgaggtctccccctggagcyacccagaacacatccaggccctacc

SEQ ID NO: 25 - DNA, 52 bp, Artificial Sequence
  Context sequence of SNP rs6962352
gggtagcagtggctgtccctgtgggtraagtcacctgaccagccactgtgag

SEQ ID NO: 26 - DNA, 52 bp, Artificial Sequence
  Context sequence of SNP rs7021928
gaactgtagcaggttctttgacatgtrttattttatatcacaacaagcagag

SEQ ID NO: 27 - DNA, 52 bp, Artificial Sequence
  Context sequence of SNP rs712886
tagtgaataagagatagacttggtcaycaaggagcaatagcttagtgagaga

SEQ ID NO: 28 - DNA, 52 bp, Artificial Sequence
  Context sequence of SNP rs7172184
agatctagctctctcaggcacaaacayccagatattttgtgatagaaggaaa

SEQ ID NO: 29 - DNA, 52 bp, Artificial Sequence
  Context sequence of SNP rs7225320
tcccctttcttaaggatacagactttyatcagcagtgatcactcattcacca

SEQ ID NO: 30 - DNA, 52 bp, Artificial Sequence
  Context sequence of SNP rs7534723
tgtactcttgcatgaaaccaggagaargttttacttggtttgctaaactttg

SEQ ID NO: 31 - DNA, 52 bp, Artificial Sequence
  Context sequence of SNP rs893109
aatatgcgaactttcacttaaaaagtrgggaaaatataggatctctgaatgc

SEQ ID NO: 32 - DNA, 52 bp, Artificial Sequence
  Context sequence of SNP rs9307866
tagtgcaagagggggatgtttggtatygtggattgcacagtgaccttgtttt

SEQ ID NO: 33 - DNA, 52 bp, Artificial Sequence
  Context sequence of SNP rs9370809
ttggagggcatgctggttgcaaccctyttattctaataaggaactggtttgg

SEQ ID NO: 34 - DNA, 52 bp, Artificial Sequence
  Context sequence of SNP rs957910
cctcctgacagtgttgtgtcctgtaaytgaaagaggattgcatctgcactca

SEQ ID NO: 35 - DNA, 52 bp, Artificial Sequence
  Context sequence of SNP rs9837484
accgacatcagtggtccattcagtggrccattaatgttgcctgacattaagt

SEQ ID NO: 36 - DNA, 52 bp, Artificial Sequence
  Context sequence of SNP rs9940922
tgtagcctccagggttgctgtggaagrgaaagagagagcaaggagggtcttg

SEQ ID NO: 37 - DNA, 52 bp, Artificial Sequence
  Context sequence of SNP rs8123323
tgatttgaaccgtcaataccaaccccytaaccccagtaaaaaaaaaaacagc

SEQ ID NO: 38 - DNA, 52 bp, Artificial Sequence
  Context sequence of SNP rs12514116
tggggcctcctggtgctcctcagaatyacgtgctctgggcaggaaaaaggtg

SEQ ID NO: 39 - DNA, 52 bp, Artificial Sequence
  Context sequence of SNP rs12925135
tttctaatccttacctctcagagggaygattatgagaaggaaatgaactatc

SEQ ID NO: 40 - DNA, 52 bp, Artificial Sequence
  Context sequence of SNP rs2663327
cttgtctctgccctgaagcacagccaygtgggtctgagaatcccctacttcc

SEQ ID NO: 41 - DNA, 52 bp, Artificial Sequence
  Context sequence of SNP rs2367910
tttttaaatctttttctttgtgaacamgtaaattgaaatgaaaagctgagta

SEQ ID NO: 42 - DNA, 52 bp, Artificial Sequence
  Context sequence of SNP rs16868972
aacatcacactatcaataataaggttkaaaggcctcatgggaaaagtccttg

SEQ ID NO: 43 - DNA, 52 bp, Artificial Sequence
  Context sequence of SNP rs12535987
taagttttgtgggcactattgataaayaaaatgaaagataaagacaaatgaa

SEQ ID NO: 44 - DNA, 52 bp, Artificial Sequence
  Context sequence of SNP rs723811
ctcacaattcaatgttttagcatataycatcaggcaaaactatcaattttga

SEQ ID NO: 45 - DNA, 52 bp, Artificial Sequence
  Context sequence of SNP rs11942354
gtggcggtgattcccaggtctggttgrtcaagactgcaatgcacaacaggaa

SEQ ID NO: 46 - DNA, 52 bp, Artificial Sequence
  Context sequence of SNP rs6923644
ggaagtaattgtattgcattaatcagkaccattatttagtattggacatttc

SEQ ID NO: 47 - DNA, 52 bp, Artificial Sequence
  Context sequence of SNP rs1556060
ctcactcaacaggtttcaatgggggarcaaataacaatacgcaaggttaata

SEQ ID NO: 48 - DNA, 52 bp, Artificial Sequence
  Context sequence of SNP rs1432443
ccttgtaacactacagaaggatatgtkaggattagagaattttagcactgga

SEQ ID NO: 49 - DNA, 52 bp, Artificial Sequence
  Context sequence of SNP rs220836
atatattttacagtagttgtcaatctrttttccagtttttctggtacttttt

SEQ ID NO: 50 - DNA, 52 bp, Artificial Sequence
  Context sequence of SNP rs7974275
taatgtaacaaccaactggtctccatktccttataggattaaaagctattaa

SEQ ID NO: 51 - DNA, 52 bp, Artificial Sequence
  Context sequence of SNP rs12275631
atatagtgagaaatattactgatgagyggactgaaactgttcattgcatatt

SEQ ID NO: 52 - DNA, 52 bp, Artificial Sequence
  Context sequence of SNP rs298542
caacgggtaggaaaggacagttggttyagtgcttgctcatgttagccctgta

SEQ ID NO: 53 - DNA, 52 bp, Artificial Sequence
  Context sequence of SNP rs6063144
actgccgtaaatcactgactttgagcytcagcttccctgtctgtaaaaacac

SEQ ID NO: 54 - DNA, 52 bp, Artificial Sequence
  Context sequence of SNP rs2820100
atgtcattagtctttgaccaatatttmtccagtccttatccagccccagttc

SEQ ID NO: 55 - DNA, 52 bp, Artificial Sequence
  Context sequence of SNP rs7075400
gtttggggaatatgtttttagaaatayacatgccatatgtgaggctatagaa

SEQ ID NO: 56 - DNA, 52 bp, Artificial Sequence
  Context sequence of SNP rs946630
tctttggggaactgaaagaagccctgragaacacaatatacaatggcacacc

SEQ ID NO: 57 - DNA, 52 bp, Artificial Sequence
  Context sequence of SNP rs10500866
cagagctgggcatatacagtaggagaygtttgctatattttaggtaattaat

SEQ ID NO: 58 - DNA, 52 bp, Artificial Sequence
  Context sequence of SNP rs6872664
tgcttttctgaactaggatcagatctytccagcctaaagtccctccactttc

SEQ ID NO: 59 - DNA, 52 bp, Artificial Sequence
  Context sequence of SNP rs2278556
ttacgtgcctatcatccagctttgtarcatcttaacattatgccgtacttgc

SEQ ID NO: 60 - DNA, 52 bp, Artificial Sequence
  Context sequence of SNP rs1861972
agaggcgaggtcaccactccctgccartggccttgcccccttcttcccccac

SEQ ID NO: 61 - DNA, 52 bp, Artificial Sequence
  Context sequence of SNP rs7766973
cccagagggtttatattttacctgcaytcctgaggatgtgtttgtgttgctt

SEQ ID NO: 62 - DNA, 52 bp, Artificial Sequence
  Context sequence of SNP rs12410279
agtactgcaaaacaggacagccatcaragattcttccctgatgacatctcag

SEQ ID NO: 63 - DNA, 52 bp, Artificial Sequence
  Context sequence of SNP rs5918
ggctcctgtcttacaggccctgcctcygggctcacctcgctgtgacctgaag

SEQ ID NO: 64 - DNA, 52 bp, Artificial Sequence
  Context sequence of SNP rs7794745
acaggtcaggacctggaaaggcctaawtgataagactaagtgtcaaaatcag

SEQ ID NO: 65 - DNA, 52 bp, Artificial Sequence
  Context sequence of SNP rs10951154
tctggtaggtagccggctgggggtggygatggtggtggtggtggtggtggtg